(19) United States
(12) Patent Application Publication
(10) Pub. No.: US 2021/0260176 A1
(43) Pub. Date: Aug. 26, 2021

(54) Methods and Compositions for Vaccinating Against Malaria

(71) Applicant: THE HENRY M. JACKSON FOUNDATION FOR THE ADVANCEMENT OF MILITARY MEDICINE, INC., Bethesda, MD (US)

(72) Inventors: Joao Carlos Aguiar, Potomac, MD (US); Keith Limbach, Gaithersburg, MD (US); Emily Smith, Silver Spring, MD (US)

(73) Assignee: United States of America as Represented by the Secretary of the Navy, Silver Spring, MD (US)

(21) Appl. No.: 16/497,244

(22) PCT Filed: Mar. 30, 2018
(86) PCT No.: PCT/US18/25510

§ 371 Date: Sep. 24, 2019

Related U.S. Application Data
(60) Provisional application No. 62/479,018, filed on Mar. 30, 2017.

(51) Int. Cl. A61K 39/015; A61P 33/06

(52) U.S. Cl. CPC A61K 39/015; A61K 2039/53; A61P 33/06

(57) ABSTRACT

The present invention provides methods and compositions for immunizing a subject against malaria.

### CROSS REFERENCE TO RELATED APPLICATIONS

The present application is a national phase entry under 35 U.S.C § 371 of International Application No. PCT/US2018/025510 filed Mar. 30, 2018, published in English, which claims priority to U.S. Provisional Application No. 62/479,018 filed Mar. 30, 2017, all of which are incorporated herein by reference.

### REFERENCE TO SEQUENCE LISTING

A computer readable text file, entitled “SequenceListing.text,” created on or about Mar. 27, 2018 with a file size of about 593 KB contains the sequence listing for this application and is hereby incorporated by reference herein in its entirety.

### BACKGROUND OF THE INVENTION

Almost all licensed vaccines are thought to mediate protection through antibody production; therefore, antigen discovery research and development has focused largely on the identification of antigens that induce protective antibodies. The availability of serum, the ease of working with antibodies, and, more recently, advances in microarray technology have facilitated these efforts. However, vaccine development for some of the most devastating infectious diseases, such as malaria, tuberculosis (TB), and HIV, has met with limited success, partially because these organisms have intracellular life cycle stages that are not targeted by antibodies, and they have developed sophisticated mechanisms to avoid clearance by host immune responses. Since T cells have been implicated in protection from these diseases, considerable efforts have been directed at developing vaccines that induce protective T cell responses. However, for infectious agents with large genomes that express many potential T cell antigens such as parasites and bacteria, many of the specific antigens that are targeted by protective CD8+ T cells are not known. Identification of the target antigens of protective T cell responses would greatly facilitate vaccine development.

Malaria killed approximately 429,000 people in 2015, most of them children in sub-Saharan Africa. Despite decades of effort, a highly effective malaria vaccine is not available. Immunization with attenuated Plasmodium sporozoites can provide high levels of protection in mice, non-human primates, and humans. Protection is mediated by CD8+ T cells, which target a set of mostly unknown pre-erythrocytic stage antigens. Activated CD8+ T cells can kill infected hepatocytes, thereby preventing blood-stage infection, which is responsible for the clinical symptoms of the disease. However, substantial delivery issues are a considerable barrier to licensure of live sporozoite-based vaccines, and broad protection against circulating strains has not been demonstrated. An alternative approach is to identify the targets of these protective CD8+ T cell responses and formulate them into a multivalent subunit vaccine designed to induce sustained T cell immunity.

The two P. falciparum sporozoite vaccines that are associated with high levels of protection in humans are radiation-attenuated sporozoites (RAS) and live sporozoites with concomitant chloroquine treatment to kill newly emerging blood-stage parasites (SPZ+CQ). Immunization with RAS leads to infection of hepatocytes and expression of a set of early liver-stage genes, but these attenuated sporozoites do not develop into late liver and blood stages. In BALB/c mice, the protective T cell response following vaccination with RAS is dominated by CD8+ T cells specific for the major surface protein on the sporozoite, the circumsporozoite protein (CSP), although T cell responses specific for other antigens can also contribute to protection. In humans, T cell responses specific for several antigens have been observed following RAS immunization. In contrast to RAS, vaccination with SPZ+CQ allows expression of the full repertoire of liver-stage genes and replication of the parasite in hepatocytes. Unlike RAS, where protection requires approximately 1,000 bites from infected mosquitoes, SPZ+CQ can provide durable protection in volunteers with as few as 30-45 bites. This robust protection is strictly dependent on CD8+ T cells and immune response to CSP is not required, highlighting the fact that the specific antigen targets of protective immunity are not known.

Pre-erythrocytic antigens, which are expressed in the sporozoite and liver stages of the Plasmodium spp. life cycle, are particularly promising targets for malaria vaccine development, with great potential to prevent infection and transmission. The pre-erythrocytic stages of the parasitic life cycle are vulnerable to vaccine intervention because their antigens are expressed at a time when low numbers of sporozoites are transmitted by the mosquito to the human host and only a few hepatocytes become infected.

Herein is described a novel platform for the discovery of antigens that are the targets of T cell responses to infection (FIG. 1). Using this system, pre-erythrocytic antigens are identified that were targeted by CD8+ T cell responses in mice immunized with protective regimens of P. yoelii SPZ+CQ. Moreover, it is demonstrated that an antigen that recalled a high frequency of interferon gamma (IFNγ)-expressing CD8+ T cells, PY03674, provided sterile protection in mice when delivered in a DNA prime-adenovector boost regimen.

### SUMMARY OF THE INVENTION

The present invention provides methods and compositions for immunizing a subject against malaria, with the methods comprising administering an immunologically effective amount of at least one antigenic polypeptide having an amino acid sequence that is at least 90, 95 or 100% identical to an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:40, NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, NO:61, SEQ ID NO:62, SEQ ID NO:63, SEQ ID NO:64, and SEQ ID NO:65, SEQ ID NO:66, SEQ ID NO:67, SEQ ID NO:68, SEQ ID NO:69, SEQ ID NO:70, NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, NO:100, SEQ ID NO:101, SEQ ID NO:102, SEQ ID NO:103, SEQ ID NO:104, SEQ ID NO:105 and SEQ ID NO:106.

### BRIEF DESCRIPTION OF THE DRAWINGS

FIG. 1 depicts a schematic view of high-throughput Ad-array generation and antigen identification assays. The general steps involved in the generating a defined array of adenovectors and their use in antigen discovery screens using high-throughput technology are indicated.

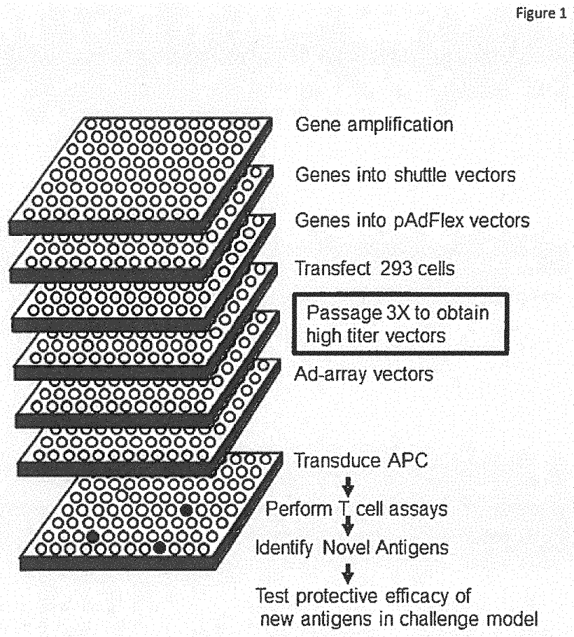

FIG. 2 depicts generation of the Ad-array. (A) >300 highly expressed malaria pre-erythrocytic genes were amplified using P. yoelii genomic DNA and gene-specific primers. The reaction products were electrophoresed on 1% agarose gels with a 1 KB ladder as shown here for a subset of these genes. The control is a pair of oligos used to amplify the E1 region of Ad5 DNA. (B) Parallel generation of two Ad-array vectors in multi-well plates. The schematic indicates two pAdFlex plasmids (pAdgPyHep17 and pAdgCMVp65), which were linearized with Pac I and transfected into 293 cells in 60 mm, 6 well, 12 well, 24, well, 48 well and 96 well plates. Following two passages in 293 cells in the same plate size, CPE was observed in all wells. Viral DNA was obtained, and PCR analysis was performed using primers that flank the expression cassette. The products of the PCR reaction were loaded into a 1% agarose gel and electrophoresed. Arrows next to AdgPyHep17 and AdgCMVp65 indicate the expected size for the PCR products. Plate sizes used to generate the recombinant adenovectors are indicated.

FIG. 3 depicts that adenovector expressed antigens are effective at recalling T cell responses from immunized mice. (A) Schema for in vitro antigen discovery. (8) Ad5 vector effectively transduces APC.A20 cells that were infected with AdGFP at the indicated MOI. The percentage of GFP positive cells was determined by FACS. (C) AdPyCSP infected APCs can recall CD8+ T cell responses from mice immunized using a PyCSP DNA vaccine. Target A20 cells were infected with various MOI of an Ad5 vector expressing PyCSP (AdPyCSP). Control targets were uninfected A20 cells, A20 cells infected with various MOI of an Ad5 vector that does not express a transgene (AdNull) and A20 cells stimulated with an immunodominant PyCSP peptide. These targets were used to stimulate splenocytes from BALB/c mice immunized with a PyCSP DNA vaccine. IFNα expressing cells were measured by ELispot. SFC (Spot Forming Cells); error bars indicate the standard error of the mean, n=3.

FIG. 4 depicts that adenovector expressed antigens are effective at recalling CD8+ T cell responses from mice immunized with protective regimens of sporozoite vaccines. Target A20 cells were infected with Ad5 vector (either triple CsCh purified AdPyCSP or unpurified cell lysate from AdPyCSP infected cells) at the indicated MOI and incubated with splenocytes from RAS immunized mice. Control targets were A20 cells infected with AdNull, AdGFP and uninfected A20 cells. (a) IFNγ+ cells were measured by ELISpot. SFC (Spot Forming Cells), (b) CD8+ IFNγ+ cells were measured by ICS staining and FACS analysis. Control targets were A20 cells infected with AdNull vectors and uninfected A20 cells. (c) Comparison of Ad-array PyCSP (AdgPyCSP) with AdPyCSP, which does not contain the recombination motifs flanking the expression cassette. CD8+ IFNγ+ cells were measured by ICS staining and FACS analysis. Controls targets were A20 cells infected with AdGFP vectors and uninfected A20 cells. (d) Dose response analysis for efficacy of SPZ+CQ vaccine regimen. (e) AdPyCSP infected cells can recall CD8+ T cell responses from mice immunized with SPZ+CQ. Target A20 cells were infected with the indicated Ad vectors and antigen specific CD8+ T cell responses were measured. Error bars indicate the standard error of the mean, n=3. The asterisks indicate statically significant differences compared with A20 controls (p<0.05 by ANOVA with Bonferroni means comparison test).

FIG. 5 depicts the identification of targets of CD8+ T cell responses induced by highly protective SPZ+CQ vaccine regimen. Splenocytes from BALB/c mice immunized with SPZ+CQ were screened for CD8+ recall responses specific for 312 pre-erythrocytic antigens. The mean of the negative controls is indicated by the horizontal line. The dotted line indicates responses that are >2 SD above the mean of the negative controls.

FIG. 6 depicts that the P. falciparum Ortholog of PY03674, PF3D7_0725100 (SEQ ID NO.: 17), Is Immunogenic in BALB/c Mice. Mice were immunized with 1×10⁹ PU of GC46.PF3D7_0725100 or a control adenovector that does not express a transgene, GC46.Null. Three weeks post-immunization, mice were euthanized and PF3D7_0725100-specific CD8+(A) and CD4+(8) T cell responses were measured by intracellular cytokine staining and flow cytometry following 4-hr stimulation using pooled overlapping 15-mer peptides.

FIG. 7 depicts identification of protective and immunogenic antigens using a matrix format. (A) Antigens (numbered 1-9) are grouped into six pools of three antigens (labeled A-F). Each antigen is present in two pools. For example, Antigen 9 is in both pools C (with 7 and 8) and F (with 3 and 6). Each pool is tested alone, and also in combination with PyCSP; therefore, each antigen is tested in four groups of mice. (B and C) CD1 mice are immunized with DNA (a pool of three antigen-expressing constructs with or without PyCSP) followed by Ad5-boost at six weeks with pooled vectors expressing the corresponding antigens. Null-immunized (4×, matching the largest dose) and naïve mice are also included as negative controls, and PyCSP alone is included as a positive control. (B) Two weeks following immunization, mice are challenged with 300 infectious P. yoelii sporozoites. Sterile protection is assessed by blood smear. (C) Two weeks following immunization, mice are challenged with 10,000 Infectious P. yoelii sporozoites by intravenous injection. Forty-two hours after challenge, mice are euthanized and livers harvest for immunological analyses and assessment of protection by quantification of liver parasite burden.

FIG. 8 depicts the use of matrices comprised of pooled adenovectors to identify T-cell antigens. Groups of 14 CD1 mice were immunized with DNA/HuAd5 vectors expressing groups of P. yoelii antigens, as described in FIG. 7. All mice were IV challenged with 300 non-lethal 17XNL P. yoelii sporozoites.

### DETAILED DESCRIPTION OF THE INVENTION

The present invention provides methods and compositions for immunizing a subject against malaria, with the methods comprising administering at least one antigenic polypeptide having an amino acid sequence that is at least 90, 95 or 100% identical to an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:40, NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, NO:61, SEQ ID NO:62, SEQ ID NO:63, SEQ ID NO:64, and SEQ ID NO:65, SEQ ID NO:66, SEQ ID NO:67, SEQ ID NO:68, SEQ ID NO:69, SEQ ID NO:70, NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, NO:100, SEQ ID NO:101, SEQ ID NO:102, SEQ ID NO:103, SEQ ID NO:104, SEQ ID NO:105 and SEQ ID NO:106.

The present invention provides the use of compositions for immunizing a subject against malaria, with the use comprising administering at least one antigenic polypeptide having an amino acid sequence that is at least 90, 95 or 100% identical to an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:40, NO:41, SEQ ID NO:42, SEQ ID NO:43, SEQ ID NO:4, SEQ ID NO:45, SEQ ID NO:46, SEQ ID NO:47, SEQ ID NO:48, SEQ ID NO:49, SEQ ID NO:50, NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, NO:61, SEQ ID NO:62, SEQ ID NO:63, SEQ ID NO:64, and SEQ ID NO:65, SEQ ID NO:66, SEQ ID NO:67, SEQ ID NO:68, SEQ ID NO:69, SEQ ID NO:70, NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, NO:100, SEQ ID NO:101, SEQ ID NO:102, SEQ ID NO:103, SEQ ID NO:104, SEQ ID NO:105 and SEQ ID NO:106.

The present invention provides use of compositions comprising at least one antigenic polypeptide having an amino acid sequence that is at least 90, 95 or 100% identical to an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:40, NO:41, SEQ ID NO:42, SEQ ID NO:43, SEQ ID NO:4, SEQ ID NO:45, SEQ ID NO:46, SEQ ID NO:47, SEQ ID NO:48, SEQ ID NO:49, SEQ ID NO:50, NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, NO:61, SEQ ID NO:62, SEQ ID NO:63, SEQ ID NO:64, and SEQ ID NO:65, SEQ ID NO:66, SEQ ID NO:67, SEQ ID NO:68, SEQ ID NO:69, SEQ ID NO:70, NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, NO:100, SEQ ID NO:101, SEQ ID NO:102, SEQ ID NO:103, SEQ ID NO:104, SEQ ID NO:105 and SEQ ID NO:106 for the manufacture of a medicament for the treatment of malaria.

The polypeptides disclosed herein are or possess novel antigens, or are orthologs thereof, that display a positive reaction to at least one of two types of screening assays for antigenicity. It is possible that the polypeptides, antigenic fragments thereof and/or orthologs thereof, also display a positive reaction to additional screening assays for antigenicity. For example, as noted in the examples, the polypeptides or fragments thereof can promote or provide a positive stimulus in an antigenic screening assay comprising flow cytometry (FACS) identification of lymphocytes stimulated in vitro with splenocytes from vaccinated animals, or the polypeptides or fragments thereof can provide or promote a positive stimulus in an antigenic screening assay comprising Enzyme-linked ImmunoSpot (EliSpot) identification of lymphocytes stimulated in vitro with splenocytes from vaccinated animals. In either screening assay, modified parasites containing the polypeptides or fragments thereof are administered to an animal with a spleen, and the spenocytes are subsequently harvested and screened for their ability to stimulate production of antigenic substances, such as but not limited to interferon gamma (IFNγ), interleukin-2 (IL-2), from lymphocytes in vitro. Accordingly, the invention is directed to polypeptides or fragments thereof that promote a positive in vitro antigenic response in lymphocytes. The phrase “promoting a positive antigenic response” is used herein to mean the polypeptides or fragments thereof can cause production of antigenic substances from lymphocytes, either directly or indirectly, such as using stimulated splenocytes as described above.

In one embodiment, the polypeptides disclosed herein are novel antigens, or the orthologs thereof, that have also been shown to induce an “antibody response” and/or a “cellular immune response” in mice immunized with radiation-attenuated sporozoites (RAS) from Plasmodium yoelii. Accordingly, the present invention provides methods of inducing an antibody response in a subject in need thereof comprising administering at least one of the polypeptides or the antigenic fragment thereof to a subject capable of producing an antibody response. The present invention also provides methods of inducing a cellular immune response in a subject in need thereof comprising administering at least one of the polypeptides or the antigenic fragment thereof to the subject.

As used herein, an “antibody response” is used as it is in the art. Namely, an antibody response occurs when a subject's immune system produces antibodies that bind specifically to an antigen upon being exposed to the antigen. The antibodies may be free in the subject's blood plasma, or the antibodies may be membrane-bound, which are often referred to as “B cell receptors” (BCRs). An antibody response, as used herein, may include production of free antibodies found in blood, tissue or other body fluids, or the antibody response may include production of membrane-bound antibodies, or both.

As used herein a “cellular immune response” or “cell mediated immunity” is an immune response in a subject that does not involve antibodies. In general, a cellular immune response includes activation of specific cell types, such as but not limited to phagocytes, and T cells, as well as release of various cytokines from immune cells. Examples of cytokines that are expressed or released during a cell-mediated immune response include but are not limited to interleukin 1 (IL-1), IL-6, IL-12, IL-16, tumor necrosis factor alpha (TNFα), interferon alpha (IFNα), IFN beta (IFNβ), IFN gamma (IFNγ), transforming growth factor beta (TGFβ), IL-4, IL-10 and IL-13.

As used herein, the terms “protein” and “peptide” are used interchangeably and simply used to denote at least a polymer, branched or unbranched, of amino acid residues. As used herein, the term “isolated,” when used in conjunction with proteins and nucleic acids, is used to indicate that the proteins or nucleic acids are present in a form in which the protein does not naturally occur. For example, the antigenic proteins of the present application are proteins that naturally occur in P. falciparum and/or P. yoelii.

Of course, the isolated antigenic proteins or fragments described herein can be purified or substantially purified. As used herein, the term “purified” when used in reference to a protein or nucleic acid, means that the concentration of the molecule being purified has been increased relative to other molecules associated with it in its natural environment, or environment in which it was produced, found or synthesized. One of skill in the art would recognize that these “other molecules” might include proteins, nucleic acids, lipids and sugars but generally do not include water, solvents, buffers, and reagents added to maintain the integrity or facilitate the purification of the molecule being purified. For example, even if a protein is diluted with an aqueous solvent during affinity chromatography, the proteins are purified by this chromatography if other naturally associated molecules do not bind to the column and are separated from the subject proteins. According to this definition, a substance may be 5% or more, 10% or more, 20% or more, 30% or more, 40% or more, 50% or more, 60% or more, 70% or more, 80% or more, 90% or more, 95% or more, 98% or more, 99% or more, or 100% pure when considered relative to its contaminants.

The term “fragment,” when used in connection with a protein, is used to mean a peptide that contains a sequence of contiguous amino acids taken from the full length or mature antigenic proteins. In specific embodiments, the antigenic protein fragments of the present invention comprise or alternatively consist of sequences of contiguous amino acids that are about 0.01 to 0.05, 0.1 to 0.5, 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95 or about 95 to 100 percent of the full length amino acid sequences disclosed herein.

The fragments of the antigenic proteins may or may not possess similar antigenicity as the full length antigenic proteins. In one embodiment, the fragments of the present invention are antigenic. In another embodiment, the fragments of the present invention are immunogenic. For example, the polypeptides of the invention may be immunologically cross-reactive and may be capable of eliciting in an animal an immune response to P. falciparum, P. vivax or P. yoelii, or infected cells thereof or antigen presenting cells expressing P. falciparum or P. yoelii antigens and/or are able to be bound by anti-protein antibodies. As used herein the term “antigenic” refers to a substance such as a peptide or nucleic acid to which an antibody or T-cell receptor specifically binds. The term “immunogenic” refers to a peptides ability to elicit at least a partial cellular immune response or antibody response. One of skill in the art readily understands the difference between an “antigenic response” and an “immunogenic response” as used herein.

As used herein, the terms “correspond(s) to” and “corresponding to,” as they relate to sequence alignment, are intended to mean enumerated positions within a reference protein, e.g., SEQ ID NO:17, and those positions in a modified protein that align with the positions on the reference protein. Thus, when the amino acid sequence of a subject protein is aligned with the amino acid sequence of a reference protein, the amino acids in the subject sequence that “correspond to” certain enumerated positions of the reference sequence are those that align with these positions of the reference sequence, but are not necessarily in these exact numerical positions of the reference sequence. Methods for aligning sequences for determining corresponding amino acids between sequences are described herein.

The amino acid residues of the antigenic proteins of the present invention may or may not be modified such as, but not limited to, addition of functional or non-functional groups such a but not limited to, acetyl groups, hydroxyl groups, carboxyl groups, carbohydrate groups (glycosylation), phosphate groups and lipid groups to name a few. Any of numerous chemical modifications may be carried out by known techniques, including but not limited to, specific chemical cleavage by cyanogen bromide, trypsin, chymotrypsin, papain, V8 protease, NaBH, acetylation, formylation, oxidation, reduction, metabolic synthesis in the presence of tunicamycin, etc.

The antigenic proteins of the present invention may or may not contain additional elements that, for example, may include but are not limited to regions to facilitate purification. For example, “histidine tags” (“his tags”) or “lysine tags” may be appended or “fused” to the antigenic proteins to create “antigenic fusion proteins.” Examples of histidine tags include, but are not limited to hexaH, heptaH and hexaHN. Examples of lysine tags include, but are not limited to pentaL, heptal and FLAG. Such regions may be removed prior to final preparation of the antigenic proteins. Other examples of a second fusion peptide include, but are not limited to, glutathione S-transferase (GST) and alkaline phosphatase (AP).

The addition of peptide moieties to antigenic proteins, whether to engender secretion or excretion, to improve stability and to facilitate purification or translocation, among others, is a familiar and routine technique in the art and may include modifying amino acids at the terminus to accommodate the tags. For example, the N-terminus amino acid may be modified to, for example, arginine and/or serine to accommodate a tag. Of course, the amino acid residues of the C-terminus may also be modified to accommodate tags. One particularly useful fusion protein comprises a heterologous region from immunoglobulin that can be used solubilize proteins.

Other types of fusion proteins provided by the present invention include but are not limited to, fusions with secretion signals and other heterologous functional regions. Thus, for instance, a region of additional amino acids, particularly charged amino acids, may be added to the N-terminus of the antigenic proteins to improve stability and persistence in the host cell, during purification or during subsequent handling and storage.

Another particular example of fusion polypeptides of the invention includes an antigenic polypeptide, fragment or variant thereof fused to a polypeptide having adjuvant activity, such as the subunit 8 of either cholera toxin or E. coli heat labile toxin. Another particular example of a fusion polypeptide encompassed by the invention includes an antigenic polypeptide fused to a cytokine, such as, but not limited to, IL-2, IL-4, IL-10, IL-12, or interferon. An antigenic polypeptide of the invention can be fused to the N- or C-terminal end of a polypeptide having adjuvant activity. Alternatively, an antigenic polypeptide of the invention can be fused within the amino acid sequence of the polypeptide having adjuvant activity.

Also, in one embodiment, the antigenic polypeptides, and fusions thereof, may comprise sequences that form one or more epitopes of a native P. falciparum and/or P. yoelii polypeptides that elicit bactericidal or opsonizing antibodies and/or CD8⁺ T cells. Such antigenic polypeptides may be identified by their ability to generate antibodies and/or CD8⁺ T cells that kill cells infected with P. falciparum and/or P. yoelii.

The present invention provides antibodies that specifically bind to one or more of the antigenic peptides of the present invention. For the production of such antibodies, isolated or purified preparations of an antigenic polypeptide of the present invention can be used as an immunogen in an immunogenic composition. The same immunogen can be used to immunize mice for the production of hybridoma lines that produce monoclonal antibodies.

In other embodiments, the antigenic polypeptides of the present invention are used as immunogens. The peptides may be produced by protease digestion, chemical cleavage of isolated or purified polypeptide, chemical synthesis or by recombinant expression, after which they are then isolated or purified. Such isolated or purified peptides can be used directly as immunogens. In particular embodiments, useful peptide fragments are 8 or more amino acids in length.

Useful immunogens may also comprise such peptides conjugated to a carrier molecule, such as a carrier protein. Carrier proteins may be any commonly used in immunology, include, but are not limited to, bovine serum albumin (BSA), chicken albumin, keyhole limpet hemocyanin (KLH), tetanus toxoid, synthetic T cell epitopes and the like.

In further embodiments, useful immunogens for eliciting antibodies of the invention comprise mixtures of two or more of any of the above-mentioned individual immunogens.

Immunization of animals with the immunogens described herein, for example in humans, rabbits, rats, ferrets, mice, sheep, goats, cows or horses, can be performed following procedures well known to those skilled in the art, for purposes of obtaining antisera containing polyclonal antibodies or hybridoma lines secreting monoclonal antibodies.

Monoclonal antibodies can be prepared by standard techniques, given the teachings contained herein. Such techniques are disclosed, for example, in U.S. Pat. Nos. 4,271,145 and 4,196,265, which are incorporated by reference. Briefly, an animal is immunized with the immunogen. Hybridomas are prepared by fusing spleen cells from the immunized animal with myeloma cells. The fusion products are screened for those producing antibodies that bind to the immunogen. The positive hybridomas clones are isolated, and the monoclonal antibodies are recovered from those clones.

Immunization regimens for production of both polyclonal and monoclonal antibodies are well known in the art. The immunogen may be injected by any of a number of routes, including subcutaneous, intravenous, intraperitoneal, intradermal, intramuscular, mucosal (e.g., nasal, vaginal, rectal), or a combination of these. The immunogen may be injected in soluble form, aggregate form, attached to a physical carrier, or mixed with an adjuvant, using methods and materials well known in the art. The antisera and antibodies may be purified using column chromatography methods well known to those of skill in the art.

The antibodies of the invention, including but not limited to those that are cytotoxic, cytostatic, or neutralizing, may be used in passive immunization to prevent or attenuate P. falciparum and/or P. yoelii infections of animals, including humans. As used herein, a cytotoxic antibody is one that enhances opsonization and/or complement killing of the protozoan bound by the antibody. As used herein, neutralizing antibody is one that reduces the infectivity of the P. falciparum and/or P. yoelii and/or blocks binding of P. falciparum, P. vivax and/or P. yoelii to a target cell. An effective concentration of polyclonal or monoclonal antibodies raised against the immunogens of the invention may be administered to a host to achieve such effects. The exact concentration of the antibodies administered will vary according to each specific antibody preparation, but may be determined using standard techniques well known to those of ordinary skill in the art. Administration of the antibodies may be accomplished using a variety of techniques, including but not limited to those described herein.

The term “antibodies” is intended to include all forms, such as but not limited to polyclonal, monoclonal, purified IgG, IgM, or IgA antibodies and fragments thereof, including but not limited to antigen binding fragments such as Fv, single chain Fv (scFv), F(ab)2, Fab, and F(ab)′ fragments, single chain antibodies as disclosed in U.S. Pat. No. 4,946,778 (incorporated by reference), as well as complementary determining regions (CDR) as disclosed in Verhoeyen and Winter, in Molecular Immunology 2ed., by B. D. Hames and D. M. Glover, IRL Press, Oxford University Press, 1996, at pp. 283-325 (incorporated by reference).

Further aspects of the invention include chimeric and/or humanized antibodies (U.S. Pat. Nos. 5,225,539; 5,585,089; and 5,530,101; all of which are incorporated by reference) in which one or more of the antigen binding regions of the antibody is introduced into the framework region of a heterologous (e.g. human) antibody. The chimeric or humanized antibodies of the invention are less antigenic in humans than non-human antibodies but have the desired antigen binding and other activities, including but not limited to neutralizing activity, cytotoxic activity, opsonizing activity or protective activity.

In one aspect of the invention, the antibodies of the invention are human antibodies. Human antibodies may be isolated, for example, from human immunoglobulin libraries (see, e.g., PCT publications WO 9846645, WO 9850433, WO 9824893, WO 9816054, WO9634096, WO 9633735, and WO 9110741, all of which are incorporated by reference) by, for example, phage display techniques (see, e.g., PCT publications WO 9002809; WO 9110737; WO 9201047; WO 9218619; WO 9311236; WO 9515982; WO 9520401 and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety. Human antibodies may also be generated from animals transgenic for one or more human immunoglobulin and that do not express endogenous immunoglobulins, see, e.g., U.S. Pat. No. 5,939,598, which is incorporated by reference. Human antibodies may also be generated as described in U.S. Patent Application No. 20130291134 which is herein incorporated by reference.

The invention also provides polynucleotides that code for the isolated antigenic proteins disclosed herein. The nucleic acids of the invention can be DNA or RNA, for example, mRNA. The nucleic acid molecules can be double-stranded or single-stranded; single stranded RNA or DNA can be the coding, or sense, strand or the non-coding, or antisense, strand. In particular, the nucleic acids may encode any of the antigenic proteins disclosed herein, as well as variants thereof. Of course, the nucleic acids of the present invention may encode additional elements, such as his tags and the like. For example, the nucleic acids of the invention would include those that encode any of the antigenic proteins and variants thereof that are also contain a glutathione-S-transferase (GST) fusion protein, poly-histidine (e.g., Hiss), poly-HN, poly-lysine, etc. If desired, the nucleotide sequences can include additional non-coding sequences such as non-coding 3′ and 5′ sequences (including regulatory sequences, for example).

Nucleic acids encoding the antigenic polypeptides of the present invention may be produced by methods well known in the art. In one aspect, nucleic acids encoding the antigenic polypeptides can be derived from polypeptide coding sequences by recombinant DNA methods known in the art. For example, the coding sequence of an antigenic polypeptide may be altered by creating amino acid substitutions that will not affect the immunogenicity of the antigenic polypeptide or which may improve its immunogenicity, such as conservative or semi-conservative substitutions as described above. Various methods may be used, including but not limited to, oligonucleotide directed, site specific mutagenesis. This and other techniques known in the art may be used to create single or multiple mutations, such as replacements, insertions, deletions, and transpositions, for example, as described in Botstein (1985) Science 229:1193-1210 which is incorporated by reference.

The identified and isolated DNA encoding the antigenic polypeptides of the present invention can be inserted into an appropriate cloning vector. A large number of vector-host systems known in the art may be used. The term “host” or “host cell” as used herein refers to either in vivo in an animal or in vitro in mammalian cell cultures.

The present invention also comprises vectors containing the nucleic acids encoding the antigenic proteins of the present invention. As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. An expression vector is one into which a desired DNA sequence may be inserted by restriction and ligation such that it is operably joined to regulatory sequences and may be expressed as an RNA transcript. Vectors may further contain one or more marker sequences suitable for use in the identification and selection of cells which have been transformed or transfected with the vector. Markers include, for example, genes encoding proteins which increase or decrease either resistance or sensitivity to antibiotics or other compounds, genes which encode enzymes whose activities are detectable by standard assays known in the art (e.g., f-galactosidase or alkaline phosphatase), and genes which visibly affect the phenotype of transformed or transfected cells, hosts, colonies or plaques. Examples of vectors include but are not limited to those capable of autonomous replication and expression of the structural gene products present in the DNA segments to which they are operably joined.

In certain respects, the vectors to be used are those for expression of polynucleotides and proteins of the present invention. Generally, such vectors comprise cis-acting control regions effective for expression in a host operatively linked to the polynucleotide to be expressed. Appropriate trans-acting factors are supplied by the host, supplied by a complementing vector or supplied by the vector itself upon introduction into the host.

A great variety of expression vectors can be used to express the proteins of the invention. Such vectors include chromosomal, episomal and virus-derived vectors, e.g., vectors derived from bacterial plasmids, from bacteriophage, from yeast episomes, from yeast chromosomal elements, from viruses such as adeno-associated virus, lentivirus, baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. All may be used for expression in accordance with this aspect of the present invention. Generally, any vector suitable to maintain, propagate or the fusion proteins in a host may be used for expression in this regard.

In select embodiments, the compositions comprise an expression vector the contains a nucleic acid that encodes at least one of the proteins of the invention, wherein the DNA expression vector is a DNA plasmid, aiphavirus, replicon, adenovirus, poxvirus, adenoassociated virus, cytomegalovirus, canine distemper virus, yellow fever virus, retrovirus, RNA replicons, DNA replicons, alphavirus replicon particles, Venezuelan Equine Encephalitis virus, Semliki Forest Virus or Sindbus Virus.

The DNA sequence in the expression vector is generally operably linked to appropriate expression control sequence(s) including, for instance, a promoter to direct mRNA transcription. Representatives of such promoters include, but are not limited to, the phage lambda PL promoter, the E. coli lac, trp and tac promoters, HIV promoters, the SV40 early and late promoters and promoters of retroviral LTRs, to name just a few of the well-known promoters. In general, expression constructs will contain sites for transcription, initiation and termination and, in the transcribed region, a ribosome binding site for translation. The coding portion of the mature transcripts expressed by the constructs will include a translation initiating AUG at the beginning and a termination codon (UAA, UGA or UAG) appropriately positioned at the end of the polypeptide to be translated.

In addition, the constructs may contain control regions that regulate, as well as engender expression. Generally, such regions will operate by controlling transcription, such as repressor binding sites and enhancers, among others.

Vectors for propagation and expression generally will include selectable markers. Such markers also may be suitable for amplification or the vectors may contain additional markers for this purpose. In this regard, the expression vectors may contain one or more selectable marker genes to provide a phenotypic trait for selection of transformed host cells. Preferred markers include dihydrofolate reductase or neomycin resistance for eukaryotic cell culture, and tetracycline, kanamycin or ampicillin resistance genes for culturing E. coli and other bacteria.

Promoter/enhancer elements which may be used to control expression of inserted sequences include, but are not limited to the SV40 early promoter region (Bernoist and Chambon, 1981, Nature 290:304-310), the promoter contained in the 3′ long terminal repeat of Rous sarcoma virus (Yamamoto (1980) Cell 22:787-797), the herpes thymidine kinase promoter (Wagner (1981) Proc. Natl. Acad. Sci. U.S.A. 78:1441-1445), the regulatory sequences of the metalothionein gene (Brinster (1982) Nature 296:39-42) for expression in animal cells, the promoters of lactamase (Villa-Kamaroff (1978) Proc. Natl. Acad. Sci. U.S.A. 75:3727-3731), tac (DeBoer (1983) Proc. Natl, Acad. Sci. U.S.A. 80:21-25), or trc for expression in bacterial cells (see also “Useful proteins from recombinant bacteria” in Scientific American, 1980, 242:74-94), the nopaline synthetase promoter region or the cauliflower mosaic virus 35S RNA promoter (Gardner (1981) Nucl. Acids Res. 9:2871), and the promoter of the photosynthetic enzyme ribulose biphosphate carboxylase (Herrera-Estrella (1984) Nature 310:115-120) for expression in plant cells; Gal4 promoter, the ADC (alcohol dehydrogenase) promoter, PGK (phosphoglycerol kinase) promoter, alkaline phosphatase promoter for expression in yeast or other fungi. The entire teachings of any reference referred to herein are incorporated by reference herein as if fully set forth herein.

Any method known in the art for inserting DNA fragments into a vector may be used to construct expression vectors containing an antigenic polypeptide encoding nucleic acid molecule comprising appropriate transcriptional/translational control signals and the polypeptide coding sequences. These methods may include in vitro recombinant DNA and synthetic techniques and in vivo recombination.

Commercially available vectors for expressing heterologous proteins in bacterial hosts include but are not limited to pZERO, pTrc99A, pUC19, pUC18, pKK223-3, pEX1, pCAL, pET, pSPUTK, pTrxFus, pFastBac, pThioHis, pTrcHis, pTrcHis2, and pLEx. For example, the phage in lambda GEM™-11 may be utilized in making recombinant phage vectors which can be used to transform host cells, such as E. coli LE392. In a preferred embodiment, the vector is pQE30 or pBAD/ThioE, which can be used transform host cells, such as E. coli.

The invention also provides for host cells comprising the nucleic acids and vectors described herein. A variety of host-vector systems may be utilized to express the polypeptide-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA, or cosmid DNA, plant cells or transgenic plants.

Hosts that are appropriate for expression of nucleic acid molecules of the present invention, fragments, analogues or variants thereof, may include E. coli, Bacillus species, Haemophilus, fungi, yeast, such as Saccharomyces, Pichia, Bordetella, or Candida, or the baculovirus expression system.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered antigenic polypeptides may be controlled. Furthermore, different host cells have characteristic and specific mechanisms for the translational and post-translational processing and modification of proteins. Appropriate cell lines or host systems can be chosen to ensure the desired modification and processing of the foreign protein expressed.

Once a suitable host system and growth conditions are established, recombinant expression vectors can be propagated and prepared in quantity. Upon expression, a recombinant polypeptide of the invention is produced and can be recovered in a substantially purified from the cell paste, the cell extract or from the supernatant after centrifugation of the recombinant cell culture using techniques well known in the art.

For instance, the recombinant polypeptide can be purified by antibody-based affinity purification, preparative gel electrophoresis, or affinity purification using tags (e.g., 6× histidine tag) included in the recombinant polypeptide.

The present invention also provides therapeutic and prophylactic compositions, which may be antigenic compositions or immunogenic compositions, including vaccines, for use in the treatment or prevention (reducing the likelihood) of P. falciparum, P. vivax and/or P. yoelii infections in human subjects (patients). The immunogenic compositions include vaccines for use in humans. The antigenic and immunogenic, compositions of the present invention can be prepared by techniques known to those skilled in the art and comprise, for example, an immunologically effective amount of any of the antigenic proteins or fragments thereof, disclosed herein, optionally in combination with or fused to or conjugated to one or more other immunogens, including lipids, phospholipids, carbohydrates, lipopolysaccharides, inactivated or attenuated whole organisms and other proteins, of P. falciparum and/or P. yoelii origin or other bacterial origin, a pharmaceutically acceptable carrier, optionally an appropriate adjuvant, and optionally other materials traditionally found in vaccines.

In one embodiment, the invention provides a cocktail vaccine comprising several antigens, which has the advantage that immunity against one or several strains of a single pathogen or one or several pathogens can be obtained by a single administration. Examples of other immunogens include, but are not limited to, those used in the known DPT vaccines, HMW protein of C. trachomatis or fragments thereof, MOMP of C. trachomatis or fragments thereof, or PMPH or HtrA of C. trachomatis or fragments thereof (preferably epitope containing fragments), entire organisms or subunits therefrom of Chlamydia, Neisseria, HIV, Haemophilus influenzae, Moraxella catarrhalis, Human papilloma virus, Herpes simplex virus, Haemophilus ducreyi, Treponema palladium, Candida albicans and Streptococcus pneumoniae, etc.

The term “immunogenic amount” or “immunologically effective amount” is used herein to mean an amount sufficient to induce an immune response. In one embodiment, the immunogenic composition is one that elicits an immune response sufficient to prevent or reduce the likelihood of P. falciparum and/or P. yoelii infections or to attenuate the severity of any preexisting or subsequent P. falciparum and/or P. yoelii infection. An immunogenic amount of the immunogen to be used in the vaccine is determined by means known in the art in view of the teachings herein. The exact concentration will depend upon the specific immunogen to be administered, but can be determined by using standard techniques well known to those skilled in the art for assaying the development of an immune response.

In one non-limiting embodiment of the invention, an effective amount of a composition of the invention produces an elevation of antibody titer after administration. In another, more specific embodiment of the invention, approximately 0.01 to 2000 μg, or 0.1 to 500 μg, or 50 to 250 μg of the protein administered is to a host. Compositions which induce CD8⁺ T cell responses which are bactericidal or reactive with host cells infected with P. falciparum and/or P. yoelii are also an aspect of the invention. Additional compositions comprise at least one adjuvant.

The combined immunogen and carrier or diluent may be an aqueous solution, emulsion or suspension or may be a dried preparation. Appropriate carriers are known to those skilled in the art and include stabilizers, diluents, and buffers. Suitable stabilizers include carbohydrates, such as sorbitol, lactose, mannitol, starch, sucrose, dextran, and glucose, and proteins, such as albumin or casein. Suitable diluents include saline, Hanks Balanced Salts, and Ringers solution. Suitable buffers include an alkali metal phosphate, an alkali metal carbonate, or an alkaline earth metal carbonate. In select embodiments, the composition of the invention is formulated for administration to humans.

The pharmaceutical and immunogenic compositions, including vaccines, of the invention are prepared by techniques known to those skilled in the art, given the teachings contained herein. Generally, an immunogen is mixed with the carrier to form a solution, suspension, or emulsion. One or more of the additives discussed herein may be added in the carrier or may be added subsequently. The vaccine preparations may be desiccated or lyophilized, for example, by freeze drying or spray drying for storage or formulations purposes. They may be subsequently reconstituted into liquid vaccines by the addition of an appropriate liquid carrier or administered in dry formulation using methods known to those skilled in the art, particularly in capsules or tablet forms.

Immunogenic, antigenic, pharmaceutical and vaccine compositions may further contain one or more auxiliary substance, such as wetting or emulsifying agents, pH buffering agents, or adjuvants to enhance the effectiveness thereof. Immunogenic, antigenic, pharmaceutical and vaccine compositions may be administered to fish, birds, humans or other mammals, including ruminants, rodents or primates, by a variety of administration routes, Including parenterally, intradermally, intraperitonealy, subcutaneously or intramuscularly.

Alternatively, the immunogenic, antigenic, pharmaceutical and vaccine compositions formed according to the present invention, may be formulated and delivered in a manner to evoke an immune response at mucosal surfaces. Thus, the immunogenic, antigenic, pharmaceutical and vaccine compositions may be administered to mucosal surfaces by, for example, the nasal, oral (intragastric), ocular, bronchiolar, intravaginal or intrarectal routes. Alternatively, other modes of administration including suppositories and oral formulations may be desirable. For suppositories, binders and carriers may include, for example, polyalkalene glycols or triglycerides. Oral formulations may include normally employed incipients such as, for example, pharmaceutical grades of saccharine, cellulose and magnesium carbonate. These compositions can take the form of microspheres, solutions, suspensions, tablets, pills, capsules, sustained release formulations or powders and contain about 0.001 to 95% of an antigenic protein. Some dosage forms may contain 50 μg to 250 μg of an antigenic protein. The immunogenic, antigenic, pharmaceutical and vaccine compositions are administered in a manner compatible with the dosage formulation, and in such amount as will be therapeutically effective, protective or immunogenic. The compositions may optionally comprise an adjuvant.

Further, the immunogenic, antigenic, pharmaceutical and vaccine compositions may be used in combination with or conjugated to one or more targeting molecules for delivery to specific cells of the immune system and/or mucosal surfaces. Some examples include but are not limited to vitamin 812, bacterial toxins or fragments thereof, monoclonal antibodies and other specific targeting lipids, proteins, nucleic acids or carbohydrates.

Suitable regimes for initial administration and booster doses are also variable, but may include an initial administration followed by subsequent administrations, such as a booster administration. The dose may also depend on the route(s) of administration and will vary according to the size of the host. The concentration of the protein in an antigenic, immunogenic or pharmaceutical composition according to the invention is in general about 0.001 to 95%, specifically about 0.01 to 5%.

The antigenic, immunogenic or pharmaceutical preparations, including vaccines, may comprise as the immunostimulating material a nucleic acid vector comprising at least a portion of the nucleic acid molecule encoding at least one antigenic protein.

A vaccine comprising nucleic acid molecules encoding one or more of the antigenic polypeptides or fragments thereof of the present invention or fusion proteins as described herein, such that the polypeptide is generated in situ is provided. In such vaccines, the nucleic acid molecules may be present within any of a variety of delivery systems known to those skilled in the art, including nucleic acid expression systems, bacterial and viral expression systems. Appropriate nucleic acid expression systems contain the necessary nucleotide sequences for expression in the patient such as suitable promoter and terminating signals. The nucleic acid molecules may be introduced using a viral expression system (e.g., vaccinia or other pox virus, alphavirus retrovirus or adenovirus) which may involve the use of non-pathogenic (defective) virus. Techniques for incorporating nucleic acid molecules into such expression systems are well known to those of ordinary skill in the art. The nucleic acid molecules may also be administered as “naked” plasmid vectors as described, for example, in Ulmer (1992) Science 259:1745-1749. Techniques for incorporating DNA into such vectors are well known to those of ordinary skill in the art. A vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods know to those skilled in the art.

Nucleic acid molecules (DNA or RNA) of the invention can be administered as vaccines for therapeutic or prophylactic purpose. Typically, a DNA molecule is placed under the control of a promoter suitable for expression in a mammalian cell. The promoter can function ubiquitously or tissue-specifically. Examples of non-tissue specific promoters include but are not limited to the early cytomegalovirus (CMV) promoter (described in U.S. Pat. No. 4,168,062) and Rous Sarcoma virus promoter (described in Norton (1985) Molec. Cell Biol. 5:281). The desmin promoter (U (1989) Gene 78:243; U (1991) J. Biol. Chem. 266:6562; and U (1993) J. Biol. Chem. 268:10401) is tissue specific and drives expression in muscle cells. More generally, useful vectors are described in, e.g., WO 9421797.

A composition of the invention can contain one or several nucleic acid molecules of the invention. It can also contain at least one additional nucleic acid molecule encoding another antigen or fragment derivative, including but not limited to, DPT vaccines, HMW protein of C. trachomatis or fragment thereof, MOMP of C. trachomatis or fragment thereof, entire organisms or subunits therefrom of Chlamydia, Neisseria, HIV Haemophilus influenzae, Moraxella catarrhalis, Human papilloma virus, Herpes simplex virus, Haemophilus ducreyi, Treponema pallidium, Candida albicans and Streptococcus pneumoniae, etc. A nucleic acid molecule encoding a cytokine, such as interleukin-1 or interleukin-12 can also be added to the composition so that the immune response is enhanced. DNA molecules of the invention and/or additional DNA molecules may be on different plasmids or vectors in the same composition or can be carried in the same plasmid or vector.

Other formulations of nucleic acid molecules for therapeutic and prophylactic purposes include sterile saline or sterile buffered saline colloidal dispersion systems, such as macromolecule complexes, nanocapsules, silica microparticles, tungsten microparticles, gold microparticles, microspheres, beads and lipid based systems including oil-in-water emulsions, micelles, mixed micelles and liposomes. A preferred colloidal system for use as a delivery vehicle in vitro and in vivo is a liposome (i.e., an artificial vesicle). The uptake of naked nucleic add molecules may be increased by incorporating the nucleic acid molecules into and/or onto biodegradable beads, which are efficiently transported into the cells. The preparation and use of such systems is well known in the art.

A nucleic acid molecule can be associated with agents that assist in cellular uptake. It can be formulated with a chemical agent that modifies the cellular permeability, such as bupivacaine (see, e.g., WO9416737).

Cationic lipids are also known in the art and are commonly used for DNA delivery. Such lipids include Lipofectin™ also known as DOTMA (N-[1-(2,3-dioeyloxy)propyl]-N,N,N-trimethylammonium chloride), DOTAP (1,2-bis(oleyloxy)-3-(trimethylammonio)propane, DDAB (dimethyldioctadecylammonium bromide), DOGS (dioctadecylamidologlycy spermine) and cholesterol derivatives such as DC-Chol (3 beta-(N—(N′,N′-dimethyl aminomethane)-carbamoyl) cholesterol. A description of these cationic lipids can be found in U.S. Pat. No. 5,283,185, WO 9115501, WO 9526356, and U.S. Pat. No. 5,527,928. Cationic lipids for DNA delivery can be used in association with a neutral lipid such as DOPE (dioleyl phosphatidylethanolamine) as described in, e.g., WO 9011092.

Other transfection facilitation compounds can be added to a formulation containing cationic liposomes. They include, e.g., spermine derivatives useful for facilitating the transport of DNA through the nuclear membrane (see, for example, WO 9318759) and membrane-permeabilizing compounds such as GALA, Gramicidine 5 and cationic bile salts (see, for example, WO 9319768).

The amount of nucleic acid molecule to be used in a vaccine recipient depends, e.g., on the strength of the promoter used in the DNA construct, the immunogenicity of the expressed gene product, the mode of administration and type of formulation. In general, a therapeutically or prophylactically effective dose from about 1 μg to about 1 mg, preferably from about 10 μg to about 800 μg and more preferably from about 25 μg to about 250 μg can be administered to human adults. The administration can be achieved in a single dose or repeated at intervals.

The route of administration can be any conventional route used in the vaccine field. As general guidance, a nucleic acid molecule of the invention can be administered via a mucosal surface, e.g., an ocular, intranasal, pulmonary, oral, intestinal, rectal, vaginal, and urinary tract surface; or via a parenteral route, e.g., by an intravenous, subcutaneous, intraperitoneal, intradermal, intra-epidermal or intramuscular route. The choice of administration will depend on the formulation that is selected. For instance, a nucleic acid molecule formulated in association with bupivacaine is advantageously administered into muscles.

Recombinant bacterial vaccines genetically engineered for recombinant expression of nucleic acid molecules encoding an antigenic protein of the present invention include Shigella, Salmonella, Vibrio cholerae, and Lactobacillus. Recombinant BCG and Streptococcus expressing one or more antigenic polypeptides can also be used for prevention or treatment of P. falciparum and/or P. yoelii infections.

Non-toxicogenic Vibrio cholerae mutant strains that are useful as a live oral vaccine are described in Mekalanos (1983) Nature 306:551 and U.S. Pat. No. 4,882,278. An effective vaccine dose of a Vibrio cholerae strain capable of expressing a polypeptide or polypeptide derivative encoded by a DNA molecule of the invention can be administered.

Attenuated Salmonella typhimurium strains, genetically engineered for recombinant expression of heterologous antigens or not and their use as oral vaccines are described in Nakayama (1988) BioTechnology 6:693 and WO9211361.

Other bacterial strains useful as vaccine vectors are described in High (1992) EMBO 11:1991; Sizemore (1995) Science 270:299 (Shigella flexneri); Medaglini (1995) Proc. Natl. Acad. Sci. US92:6868 (Streptococcus gordonii); and Flynn (1994) Cell Mol. Biol. 40:31; WO 886626; WO 900594; WO 9113157; WO 921796; and WO 0221376 (Bacille Calmette Guerin).

In genetically engineered recombinant bacterial vectors, nucleic acid molecule(s) of the invention can be inserted into the bacterial genome, carried on a plasmid, or can remain in a free state.

When used as vaccine agents, recombinant bacterial or viral vaccines, nucleic acid molecules and polypeptides of the invention can be used sequentially or concomitantly as part of a multistep immunization process. For example, a mammal can be initially primed with a vaccine vector of the invention such as pox virus or adenovirus, e.g., via the parenteral route or mucosally and then boosted several time with a polypeptide e.g., via the mucosal route. In another example, a mammal can be vaccinated with polypeptide via the mucosal route and at the same time or shortly thereafter, with a nucleic acid molecule via intramuscular route.

The antigenicity and/or immunogenicity of the peptides or fragments described herein may or may not necessarily require the use of an immunologically effective amount of an adjuvant or combination of adjuvants such as, but not limited to, alum, aluminum phosphate, aluminum hydroxide, squalene, oil-based adjuvants, virosomes, OS21, MFS9, Army Liposoma Formulation (ALF) with or without QS-21 (Genito et al, Vaccine 35:3865 (2017)), interleukin 12 (IL-12), CpG, small molecule mast cell activator (MP7), TLR7 imidaroquinoline ligand 3M-019, resquimod (R848), N-acetyl-muramyl-L-threonyl-D-isoglutamine (thr-MDP), N-acetyl-nor-muramy-L-alanyl-D-isogiutamine (CGP11637, referred toasnor-MDP), N-acetylmuramy-L-alanyl-D-isoglutaminyl-L-alanine-2-(1′-2′-dip-amitoyl-sn-glycero-3 hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80. Table I provides information on adjuvants that may be useful. Table I shows possible adjuvants and their properties. These adjuvants may be used alone or in combination to test their ability to augment the immune response towards P. falciparum and/or P. yoelii. These adjuvants are defined by their ability to drive a Th1 or Th2 response.

TABLE 1

| Adjuvant | Properties |
|---|---|
| CT | Potent mucosal adjuvant (Th2 response) |
| CpG | TLR 9 agonist (Th1 response) |
| MplA | TLR 4 agonist (Th1 response) |
| R 848 | TLR 7/8 agonist (Th1 response) |
| IL-12 | Pro-inflammatory cytokine (Th1 response) |
| ALF | Th1 |
| CT + CpG | Th2 + Th1 response |
| CT + MplA | Th2 + Th1 response |
| CT + R 848 | Th2 + Th1 response |
| CT + IL-12 | Th2 + Th1 response |
| CpG + MplA | Th1 response |
| CpG + R 848 | Th1 response |
| CpG + Pam3CSK4 | Th1 response |

immunostimulatory agents or adjuvants have been used for many years to improve the host immune responses to, for example, vaccines. Intrinsic adjuvants, such as lipopolysaccharides, normally are the components of the killed or attenuated bacteria used as vaccines. Extrinsic adjuvants are immunomodulators which are typically non-covalently linked to antigens and are formulated to enhance the host immune responses. Thus, adjuvants have been identified that enhance the immune response to antigens delivered parenterally. Aluminum hydroxide, aluminum oxide, and aluminum phosphate (collectively commonly referred to as alum) are routinely used as adjuvants in human and veterinary vaccines.

Other extrinsic adjuvants may include chemokines, cytokines (e.g., IL-2), saponins complexed to membrane protein antigens (immune stimulating complexes), pluronic polymers with mineral oil, killed mycobacteria in mineral oil, Freund's complete adjuvant, bacterial products, such as muramyl dipeptide (MDP) and lipopolysaccharide (LPS), as well as lipid A, and liposomes.

U.S. Pat. No. 6,019,982, incorporated herein by reference, describes mutated forms of heat labile toxin of enterotoxigenic E. coli (“mLT”). U.S. Pat. No. 5,057,540, incorporated herein by reference, describes the adjuvant, QS21, an HPLC purified non-toxic fraction of a saponin from the bark of the South American tree Quiliaja saponaria molina. 3D-MPL is described in Great Britain Patent 2,220,211, which is incorporated herein by reference.

U.S. Pat. No. 4,855,283, which is incorporated herein by reference, teaches glycolipid analogues including N-glycosylamides, N-glycosylureas and N-glycosylcarbamates, each of which is substituted in the sugar residue by an amino acid, as immuno-modulators or adjuvants. Lockhoff reported that N-glycosphospholipids and glycoglycerolipids are capable of eliciting strong immune responses in both herpes simplex virus vaccine and pseudorables virus vaccine. Some glycolipids have been synthesized from long chain-alkylamines and fatty acids that are linked directly with the sugars through the anomeric carbon atom, to mimic the functions of the naturally occurring lipid residues.

U.S. Pat. No. 4,258,029 granted to Moloney, incorporated herein by reference, teaches that octadecyl tyrosine hydrochloride (OTH) functions as an adjuvant when complexed with tetanus toxoid and formalin inactivated type I, II and III poliomyelitis virus vaccine. Lipidation of synthetic peptides has also been used to increase their immunogenicity.

Therefore, according to the invention, the immunogenic, antigenic, pharmaceutical, including vaccine, compositions may further comprise immune-effective amounts of an adjuvant, such as, but not limited to alum, mLT, LTR192G, QS21, RIBI DETOX™, MMPL, CpG DNA, MF59, calcium phosphate, PLG interleukin 12 (IL12), TLR7 imidazoquinoline ligand 3M-019, resquimod (R848), small molecule mast cell activator MP7, ALF (with or without QS-21), and all those listed above. The adjuvant may be selected from one or more of the following: alum, QS21, CpG DNA, PLG, IT, 3D-mPL, or Bacille Calmette-Guerine (BCG) and mutated or modified forms of the above, particularly mLT and LTR192G. The compositions of the present invention may also further comprise a suitable pharmaceutical carrier, including but not limited to saline, bicarbonate, dextrose or other aqueous solution. Other suitable pharmaceutical carriers are described in Remington's Pharmaceutical Sciences, Mack Publishing Company, a standard reference text in this field, which is incorporated herein by reference in its entirety.

Immunogenic, antigenic and pharmaceutical, including vaccine, compositions may be administered in a suitable, nontoxic pharmaceutical carrier, may be comprised in microcapsules, microbeads, and/or may be comprised in a sustained release implant.

Table 2 provides a list of antigenic proteins useful in the methods and compositions of the present invention. With respect to Table 1, the “FC” indicates flow cytometry, “ES” indicates ElisaSpot screening, P. yoelii indicates Plasmodium yoelii, P. falciparum indicates Plasmodium falciparum (isolate 3D7), and P. vivax indicates Plasmodium vivax (Sal-1).

TABLE 2
LIST OF ANTIGENIC PROTEINS

| UniProt | | | SEQ ID | | |
|---|---|---|---|---|---|
| Accession No. | Antigen Name | Source | NO. | Length | Screen |
| Q7RNQ7 | PY01758 | P. yoelii | 1 | 274 | FC |
| Q7RS41 | PY00525 | P. yoelii | 2 | 227 | FC, ES |
| Q7RK90 | PY03011 | P. yoelii | 3 | 241 | ES |
| Q7RIF0 | PY03674 | P. yoelii | 4 | 1368 | FC, ES |
| Q7RHD9 | PY04050 | P. yoelii | 5 | 1154 | FC |
| Q7RJH1 | PY03289 | P. yoelii | 6 | 297 | FC |
| Q7RML7 | PY02161 | P. yoelii | 7 | 463 | FC |
| Q7RL60 | PY02686 | P. yoelii | 8 | 218 | FC |
| Q7RJ67 | PY03396 | P. yoelii | 9 | 966 | ES |
| Q7RFZ3 | PY04558 | P. yoelii | 10 | 1121 | ES |
| Q7R985 | PY06979 | P. yoelii | 11 | 563 | ES |
| Q7RJX9 | PY03126 | P. yoelii | 12 | 347 | ES |
| Q7RKV7 | PY02793 | P. yoelii | 13 | 1488 | FC |
| Q8IJ98 | PF3D7_1030700 | P. falciparum | 14 | 257 | FC, ES |
| C0H4L2 | MAL7P1.203 | P. falciparum | 15 | 1526 | FC |
| Q8ILV3 | PF3D7_1414200 | P. falciparum | 16 | 407 | FC, ES |
| Q8IBK0 | PF3D7_0725100 | P. falciparum | 17 | 1576 | FC, ES |
| Q7RSJ8 | PY00357 | P. yoelii | 18 | 1095 | |
| Q7RQ59 | PY01244 | P. yoelii | 19 | 283 | |
| Q7RM58 | PY02329 | P. yoeiii | 20 | 499 | |
| Q7PDQ7 | PY03587 | P. yoelii | 21 | 1140 | |
| Q7RHD8 | PY04051 | P. yoelii | 22 | 967 | |
| Q7RF93 | PY04814 | P. yoelii | 23 | 335 | |
| Q7REI1 | PY05083 | P. yoelii | 24 | 603 | |
| Q7RC14 | PY05971 | P. yoelii | 25 | 548 | |
| Q7RBU9 | PY06037 | P. yoelii | 26 | 135 | |
| Q7RAM5 | PY06474 | P. yoelii | 27 | 78 | |
| Q7RAM2 | PY06477 | P. yoelii | 28 | 58 | |
| Q7RAG1 | PY06539 | P. yoelii | 29 | 2236 | |
| Q7RAE1 | PY06559 | P. yoelii | 30 | 1401 | |
| Q7R8T3 | PY07137 | P. yoelii | 31 | 1060 | |
| Q7R862 | PY07361 | P. yoelii | 32 | 319 | |
| Q7R7U4 | PY07484 | P. yoelii | 33 | 48 | |
| Q7RMF3 | PY02228 | P. yoelii | 34 | 387 | |
| Q7RKB2 | PY02989 | P. yoelii | 35 | 670 | |
| Q7REN6 | PY05028 | P. yoelii | 36 | 741 | |
| Q7RCT4 | PY05693 | P. yoelii | 37 | 304 | |
| Q7R7H8 | PY07608 | P. yoelii | 38 | 145 | |
| Q7RLY3 | PY02405 | P. yoelii | 39 | 138 | ES |
| O97302 | PF3D7_0323400 | P. falciparum | 40 | 1086 | |
| Q8I294 | PF3D7_0104500 | P. falciparum | 41 | 277 | |
| P61074 | PCNA PF13_0328 | P. falciparum | 42 | 274 | |
| Q8II84 | PF3D7_1127900 | P. falciparum | 43 | 409 | |
| C6KT88 | PF3D7_0625200 | P. falciparum | 44 | 385 | |
| Q7K6A7 | PF3D7_0518400 | P. falciparum | 45 | 229 | |
| A0A143ZXJ2 | PF3D7_0706100 | P. falciparum | 46 | 1529 | |
| Q9U0L0 | PF3D7_0407600 | P. falciparum | 47 | 1212 | |
| Q8IBA2 | PF3D7_0827000 | P. falciparum | 48 | 1289 | |
| Q8I0W7 | PF3D7_0518500 | P. falciparum | 49 | 1123 | |
| Q8IDT1 | PF3D7_1340500 | P. falciparum | 50 | 1202 | |
| Q8I4X7 | PF307_1245400 | P. falciparum | 51 | 341 | |
| Q8IK99 | PF3D7_1473900 | P. falciparum | 52 | 852 | |
| O96252 | PF3D7_0217100 | P. falciparum | 53 | 551 | |
| Q9U0M0 | PF3D7_0406600 | P. falciparum | 54 | 139 | |
| Q8IIB0 | PF3D7_1125300 | P. falciparum | 55 | 1,531 | |
| Q8ILJ3 | PF3D7_1427100 | P. falciparum | 56 | 1,320 | |
| Q8ID57 | PF3D7_1365000 | P. falciparum | 57 | 348 | |
| C0H541 | PF3D7_0916800 | P. falciparum | 58 | 49 | |
| Q8IAU4 | PF3D7_0810900.1 | P. falciparum | 59 | 345 | |
| O96209 | PF3D7_0212800 | P. falciparum | 60 | 1,224 | |
| Q8IEU1 | PF3D7_1302200 | P. falciparum | 61 | 229 | |
| Q8IB79 | PF3D7_0824500 | P. falciparum | 62 | 373 | |
| O97238 | PF3D7_0305300 | P. falciparum | 63 | 956 | |
| C0H494 | PF3D7_0407100 | P. falciparum | 64 | 333 | |
| Q8IDJ0 | PF3D7_1350900 | P. falciparum | 65 | 521 | |
| A5K7J3 | PVX_095055 | P. vivax | 66 | 1075 | |
| A5KDZ0 | PVX_111190 | P. vivax | 67 | 214 | |
| A5K9W6 | PVX_081530 | P. vivax | 68 | 282 | |
| A5K2M4 | PVX_115055 | P. vivax | 69 | 274 | |
| A5K4T9 | PVX_092005 | P. vivax | 70 | 426 | |
| A5K284 | PVX_114365 | P. vivax | 71 | 406 | |
| A5K9H4 | PVX_080325 | P. vivax | 72 | 237 | |
| A5KA67 | PVX_087845 | P. vivax | 73 | 1522 | |
| A5K187 | PVX_085740 | P. vivax | 74 | 349 | |
| A5KAM0 | PVX_000865 | P. vivax | 75 | 1157 | |
| A5K512 | PVX_089015 | P. vivax | 76 | 1181 | |
| A5KC29 | PVX_096085 | P. vivax | 77 | 1396 | |
| Q8I3S4 | PF3D7_0518600 | P. falciparum | 78 | 1276 | |
| A5K9H2 | PVX_080315 | P. vivax | 79 | 1240 | |
| A5K9H3 | PVX_080320 | P. vivax | 80 | 1006 | |
| A0A1K9YEP8 | PVX_082937 | P. vivax | 81 | 365 | |
| A5K8V6 | PVX_101165 | P. vivax | 82 | 336 | |
| A5K321 | PVX_116790 | P. vivax | 83 | 589 | |
| A5KBV3 | PVX_002685 | P. vivax | 84 | 564 | |
| A5KAN0 | PVX_000915 | P. vivax | 85 | 144 | |
| C6KSZ7 | PF3D7_0615600 | P. falciparum | 86 | 2528 | |
| A5K1Z4 | PVX_113915 | P. vivax | 87 | 2345 | |
| A5K4R4 | PVX_091885 | P. vivax | 88 | 1335 | |
| A5K0X8 | PVX_085180 | P. vivax | 89 | 1960 | |
| A5K2Q5 | PVX_115210 | P. vivax | 90 | 322 | |
| A0A1G4GV62 | PVX_099263 | P. vivax | 91 | 48 | |
| A5JZS0 | PVX_123225 | P. vivax | 92 | 351 | |
| A5KBZ4 | PVX_002890 | P. vivax | 93 | 865 | |
| A5K5K5 | PVX_089135 | P. vivax | 94 | 373 | |
| A5KB70 | PVX_119390 | P. vivax | 95 | 837 | |
| A5KAM5 | PVX_000890 | P. vivax | 96 | 321 | |
| A5K8E6 | PVX_083440 | P. vivax | 97 | 660 | |
| Q7RTC4 | PY00070 | P. yoelii | 98 | 438 | |
| O96158 | PF3D7_0206500 | P. falciparum | 99 | 1436 | |
| A5KBL3 | PVX_003755 | P. vivax | 100 | 1085 | |
| Q7RLV7 | PY02432 | P. yoelii | 101 | 149 | |
| C6S3F9 | PF3D7_1137800 | P. falciparum | 102 | 151 | |
| A5K538 | PVX_092505 | P. vivax | 103 | 154 | |
| Q7RNK9 | PY01807 | P. yoelii | 104 | 227 | |
| Q8I5L3 | PF3D7_1219900 | P. falciparum | 105 | 227 | |
| A5K001 | PVX_123635 | P. vivax | 106 | 227 | |

The examples disclosed herein are provided for illustrative purposes only and are not intended to limit the scope of the invention in any manner.

### EXAMPLES

### Example 1: Methods

For radiation-attenuated sporozoites (RAS) immunizations, 60 female BALB/c mice were immunized, via tail vein injection, with three doses of RAS (10,000, 5,000, and 5,000) at three week intervals. For generation of RAS, P. yoelii sporozoites were attenuated at 10,000 rads.

For SPZ+CQ immunizations, female BALB/c mice (n=6/group) were immunized with two administrations (one month apart) of live P. yoelii sporozoites. Various doses of sporozoites were tested (group 1=20,000, group 2=2,000, group 3=200, group 4=0). Immunized mice received a 0.1 ml intraperitoneal injection of a solution of chloroquine hydrochloride (Sigma-Aldrich) 8 mg/ml diluted in PBS, to kill newly emerging blood stage parasites, starting on the same day as sporozoite immunizations and continuing for ten consecutive days following each immunization.

For DNA-Ad5 immunizations, BALB/c mice were immunized with 100 μg of DNA vector, pcDNA3.2-Dest (Invitrogen) in a 0.1 ml volume by intramuscular immunization. Six weeks later these mice were boosted with 1×10¹⁰ pfu of Ad vector in a 0.1 ml volume. Both DNA and Ad were injected bilaterally into the tibialis anterior muscles with a 0.3 ml syringe and a 29.5 G needle (Becton Dickinson).

A20.2 J cells (ATCC) were grown in 15 ml of fresh RPMI-1640 media plus 20% FBS and 1% L-glutamine in 25 ml T-flasks. The T-flasks were kept upright and incubated in a 5% CO₂ incubator at 37° C. overnight. When the cells reached a density of 1.2-1.8×10⁶ cells/ml they were used to seed 12 well plates at a density of 5.0×10⁵ cells/well. The following day the cells were infected with AdGFP, an adenovirus vector that expresses GFP, for 2 hours in a volume of 200 μl. After infection, cells were washed with PBS, overlaid with 1 ml of fresh media and incubated at 37° C. and 5% C02 for 48 hours. The percentage of the GFP positive cells was analyzed by FACS

For the array screening, A20.2J cells were infected with 200 μl CPE lysate from each of the Ad-array vectors in 24 well plates for 2 hours. After infection, cells were washed with PBS, overlaid with 0.6 ml of fresh media and incubated at 37° C. and 5% CO₂ for 24 hours.

To screen for antigenicity, splenocytes harvested from vaccinated animals were stimulated by co-culture with infected/irradiated A20J2 cells in 96 well plates. Briefly, spleens were gently crushed using the flat end of a 3 cc or 10 cc syringe plunger, cell suspension was passed through a 70 μm filter. The splenocytes were washed twice with 0.5% FBS/10 mM HEPES/1×HBSS. To remove the red blood cells (RBC), 5 ml of RBC lysing buffer (Sigma) were added to the cell pellets, and the tubes were swirled gently to mix the cells with the buffer, then incubated for 3 minutes at room temperature. After 3 minutes, a 1:15 dilution of the samples with 0.5% FBS/10 mM HEPES/HBSS buffer was immediately performed. The cells were washed with RPMI once more, counted and diluted to 5×10⁶ cells/ml in RPMI medium.

At 24 hours after infection, A20 cells were irradiated in a Pantak X-Rad 320 irradiator at 16,666 rads. After irradiation, 1.5×10⁵ infected cells were transferred to each well of U-bottom 96-well plates preloaded with 1×10⁶ splenocytes from vaccinated or naïve mice, in triplicate, and incubated for 8 hours at 37° C. BD Golgi Plug™ (BD Bioscience) was added 1 hour into the incubation to block cytokine release. Cells were centrifuged at 1200 rpm for 5 minutes, the supernatant flicked, and the cell pellets resuspended by gentle vortexing. Live and dead cells were first stained with Live/Dead Fixable Aqua stain kit (BD Biosciences), then the cells were blocked with FC Block™ (BD Biosciences). After blocking, cell surface markers were stained with the following antibodies-(fluorochromes):CD4-eFlur-450 and CD8a-PerCP-Cy5.5 (BD Biosciences). Following separate fixation and permeabilization steps, the samples were stained intracellularly with the following antibodies-(flurochromes): IFNγ, -PE, TNF-α, APC, and IL-2—Alexa 488 (BD Biosciences). The frequency of CD4, CD8+ T cells, as well as peptide-specific IFNα and IL-2 intracellular cytokines positive T cells, was determined in an 8-color upgraded FACSCalibur™ (Becton Dickinson immunocytometry Systems) with 96 well Automated Micro-sampling System (AMS) (Cytek). Data were analyzed using Flowjow software (Trestar).

To evaluate cellular responses of mice immunized with irradiated sporozoites to novel antigens, cDNA from P. yoelii sporozoites was cloned into an adapted VR1020 plasmid (Vical) containing Gateway recombination sites (Invitrogen). VR1020 constructs encoding P. yoelii genes were transfected into the A20 cell line using AMAXA Nucleofection (Lonza) according to the manufacturer's instructions. Two million A20 cells were transfected with 5 μg of DNA, using either VR1020 encoding novel P. yoelii antigens, PyCSP, or VR1020-null. Transfection efficiency for each assay was monitored by transfection of the GFP-expressing control plasmid. Twenty-four hours post-transfection, cells were harvested and irradiated at 16,666 Rads, prior to plating for the IFN-γ ELISpot assay. Multiscreen HTS HA 96-well filter plates (Millipore) were coated with 1 μg in 100 μL of anti-mouse IFN-γ antibody done R4-6A2 in 1×PBS pH 7.4. Plates were incubated overnight at room temperature, and then washed with RPMI. Plates were then blocked with complete medium [RPMI-1640 with 25 mM HEPES and L-glutamine, supplemented with 10% heat-inactivated Fetal Calf Serum, 2 mM L-glutamine, and Penicillin-Streptomycin (Invitrogen)] for a minimum of 3 hours. Each well was plated with 400,000 splenocytes (immunized or naïve) and 100,000 transfected A20 cells. Plates were incubated for 36 hours prior to development. Cells were then discarded, and plates were washed six times with 1×PBS containing 0.05% Tween-20 using a Dynex plate washer. Each well was incubated for 3-5 hours at room temperature or overnight at 4° C. with 100 μL 2 g/mL biotinylated anti-mouse IFN-γ clone XMG1.2 (Pharmingen). Plates were then washed three times with PBS containing 0.05% Tween-20 using a Dynex plate washer. Wells were incubated with 100 μl Streptavidin-HRP (KPL) at room temperature for one hour according to the manufacturer's instructions, then washed three times with PBS-Tween as above, and then three times with PBS pH 7.4 alone. Plates were developed with 3,3′-diaminobenzidine (DAB) substrate (KPL) according to the manufacturer's instructions, and the reaction was stopped by flooding the plate with water. After drying, spots were counted using an AID ELISpot reader.

Replication-incompetent adenovirus vectors contain a deletion in one or more replication-essential genes resulting in an adenovirus vector that cannot replicate in typical host cells, including a human patient. A replication-incompetent adenovirus vector, however, can be grown in a cell line that expresses the adenovirus genes necessary for replication. For example, replication-incompetent HuAd5 vectors that contain a deletion in the HuAd5 E1 region can be grown in the 293 cell line that expresses the HuAd5 E1 region, and replication-incompetent HuAd5 vectors that contain a deletion in the HuAd5 E1, E3 and E4 regions can be grown in the 293ORF6 cell line that expresses the HuAd5 E1 and E4ORF6 regions. Two different types of replication-incompetent HuAd5 vectors were used in the methods described herein: vectors that contain deletions in the E1, E3 and E4 regions and vectors that contain a deletion in the E1 region. Replication-incompetent HuAd5 E1-, partial E3-, E4-vectors were constructed using a method in which a foreign gene was recombined into a plasmid containing the HuAd5 genome in E. coli cells. Briefly, a Plasmodium gene was cloned into a small shuttle vector downstream from a human cytomegalovirus (HCMV) immediate-early (IE) promoter and between HuAd5 flanking arms. The Plasmodium expression cassette was then recombined into a large plasmid containing the HuAd5 genome by transforming the small shuttle plasmid containing the Plasmodium expression cassette between HuAd5 flanking arms and a large plasmid containing the entire HuAd5 genome (minus HuAd5 E1, E3 and E4 regions) into a recombination-positive strain of E. coli, BJDE3. A recombinant plasmid in which the Plasmodium expression cassette has been recombined into the large HuAd5 plasmid was then identified by restriction enzyme analysis.

The large recombinant plasmid containing the Plasmodium expression cassette was then transformed into a recombination-negative strain of E. coli and isolated by standard microbiological methods. The HuAd5 sequence (containing the Plasmodium expression cassette) was liberated from the large plasmid by digestion with a restriction endonuclease. This DNA was then transfected into 293ORF6 cells. Cell lysates were serially passaged every 3-4 days until cytopathic effect (CPE) was observed. CPE is an indication that the viral vector is growing in the complementing cell line. Virus was then expanded from a single 60 mm dish to at least 10 T175 flasks. Following the final infection, the recombinant vectors were released from infected cells by 3 freeze-thaws, treated with benzonase, purified by banding on a CsCl gradient, dialyzed with a HuAd5 buffer and stored at −80° C. Particle unit (pu) titers were then determined by absorbance at 260 nm.

Replication-incompetent HuAd5 E1-vectors were generated using a site-specific recombination-based cloning method which allows for the transfer of DNA segments between different cloning vectors in vitro without the need for restriction endonucleases and ligase. The Gateway™ cloning system relies on a site-specific recombination process between bacteriophage A and E. coli. Briefly, a Plasmodium gene was cloned into a kanamycin resistant (Kmr) Gateway™ “Entry” vector between two recombination sites (attL1 and attL2). The Plasmodium gene was then recombined into a large ampicillin resistant (Apr) Gateway™ “Destination” vector that contains the entire HuAd5 genome (minus the E1 region). This “Destination” vector also contains two recombination sites (attR1 and attR2) that flank a gene for negative selection, ccdB. When the “Entry” and “Destination” vectors are combined, recombination occurs between attL1 and attR1 and between attL2 and attR2. The product of this recombination event is a large plasmid in which the Plasmodium gene was cloned into the HuAd5 genome downstream from a HCMV IE promoter. The large plasmid containing the Plasmodium expression cassette was then digested with a restriction endonuclease to liberate the HuAd5 sequence, and the DNA was transfected into 293 cells.

Cell lysates were serially passaged every 3 or 4 days until CPE is observed. Virus was expanded from a single 60 mm dish to at least 10 T175 flasks. Following the final infection, the recombinant vectors were released from infected cells by 3 freeze-thaws, treated with benzonase, purified by banding on a CsCl gradient, dialyzed with a HuAd5 buffer and stored at −80° C. Particle unit (pu) titers are then determined by absorbance at 260 nm.

Mice were immunized with a 100 μg of DNA vector expressing the specific antigen and then boosted 6 weeks later with an Ad5 vector (1×10¹ pfu) expressing the same antigen. Two weeks after the Ad5 boost, mice were challenged intravenously in the tail vein with 200 P. yoelii sporozoites using a 1 ml syringe and 26.5 G needle (Becton Dickinson).

Sporozoites were hand dissected from infected mosquito salivary glands and diluted for challenge in M199 medium containing 5% normal mouse serum (Gemini Bio-Products). The development of parasitemia was monitored over the next 2 weeks by microscopic examination of geimsa stained blood smears. Mice were considered protected if no parasites were observed in any sample at day 6, day 9 or day 14 post challenge.

### Example 2: Generation of an Array of Adenovectors that Express a Panel of Highly Expressed P. yoelii Pre-Erythrocytic Antigens

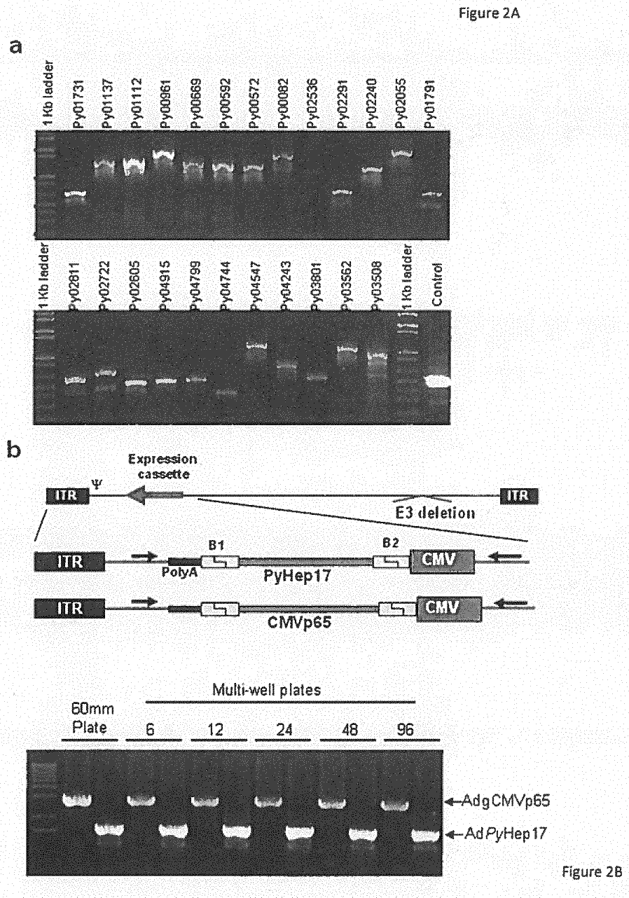

P. yoelii pre-erythrocytic genes with identifiable P. falciparum orthologs were selected for generation of an adenovector array (Ad-array) based on their level of expression in microarray and protein mass spectrometry datasets. Gene selection was made without regard to protein function or subcellular localization. In total, 312 P. yoelii genes were amplified from genomic DNA and cloned into E1/E3-deleted adenovirus type 5 (Ad5) vector genomes (FIG. 2A).

To facilitate high-throughput production of the Ad-array, the efficiency of adenovector generation was compared in multi-well plates of different sizes. The adenovector plasmid had to convert into an adenovirus vector in sufficient quantities and quality to function in the antigen screening assay. Initially, conversions were tested of two pAdFlex plasmids that expressed the P. yoelii Hep17 antigen (AdgHep17) and the cytomegalovirus p65 antigen (AdgCMVp65). These large plasmids were transfected into 293 cells in 60-mm, 6-well, 12-well, 24-well, 48-well, and 96-well plates, and the cells were passaged to increase the adenovector titer. Efficient adenovector conversion was observed in all of the wells as indicated by full cytopathic effect (CPE) at passage 2. Vector identity was verified by PCR using oligonucleotides that spanned the expression cassette (FIG. 28). Vector titers from each of the CPE wells (Table 3) demonstrated equivalent yields per infected cell. These results indicated that multiple adenovectors can be generated from pAdFlex adenovector plasmids in a parallel process in multi-well plates and that 96-well plates were suitable for the generation of the Ad-array.

TABLE 3
VECTOR YIELDS ON VARIOUS SIZE PLATES

| | |
|---|---|
| Adg.PyHEP17 | Adg.CMVp65 |

| | | | | |
|---|---|---|---|---|
| Plate Size | ffu/ml | ffu total | ffu/ml | ffu total |

| | | | | | |
|---|---|---|---|---|---|
| 60 | mm | 3.10E+08 | 3.10E+08 | 8.20E+08 | 3.44E+09 |
| 6 | well | 4.55E+08 | 9.55E+08 | 6.70E+08 | 1.41E+09 |
| 12 | well | 9.00E+08 | 7.56E+08 | 1.10E+09 | 9.246+08 |
| 24 | well | 1.33E+09 | 5.59E+08 | 1.02E+09 | 4.28E+08 |
| 48 | well | 1.38E+09 | 5.80E+07 | 1.27E+09 | 5.30E+07 |
| 96 | well | 1.26E+09 | 2.26E+07 | 1.19E+09 | 2.10E+07 |

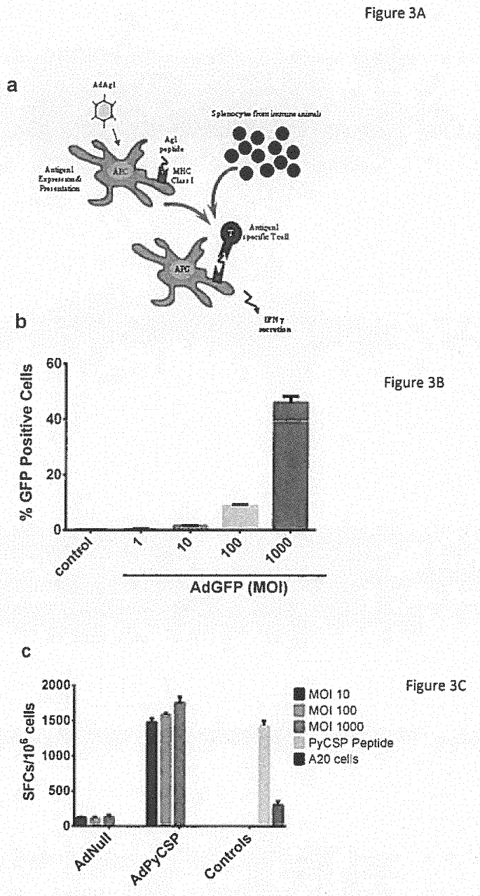

The overall design of an antigen screening system is shown in FIG. 3A. To test the elements of the screen, the MOI necessary to efficiently infect A20 cells was determined. Cells were infected with various doses of AdGFP, an Ad5 vector expressing GFP, and the percentage of infected cells was measured 48 hr post-infection (FIG. 38). MOI of 10, 100, or 1,000 focal forming units (ffus)/cell were required to infect approximately 2%, 10%, or 50% of the cells, respectively. To determine if adenovirus vectors could efficiently present antigen following infection of antigen presenting cells (APCs), we immunized BALB/c mice with a PyCSP-expressing plasmid, stimulated splenocytes from these mice with APCs infected with an Ad5 vector expressing PyCSP (AdPyCSP), and measured activated T cells by the enzyme linked immunosorbent spot (ELISpot) assay. Strong recall responses to the AdPyCSP-infected cells were observed, even at a low MOI, comparable to those generated by pulsing APCs with a peptide containing the PyCSP immunodominant epitope (FIG. 3C). Very low responses were seen in the negative controls. These results demonstrate that A20 cells (which express both major histocompatibility complex [MHC] class I and class II alleles) infected with AdPyCSP are able to present antigen to immune T cells. This process was highly efficient, as strong T cell responses were observed even at an MOI of 10, a multiplicity that resulted in transduction of approximately 2% of the target cells. Increasing the MOI resulted in substantially increased A20 cell transduction (FIG. 3B) but only marginally increased functional activity in the ELISpot assay (FIG. 3C). Thus, low-level target cell transduction is sufficient for optimal activity to detect T cell responses in the ELISpot assay.

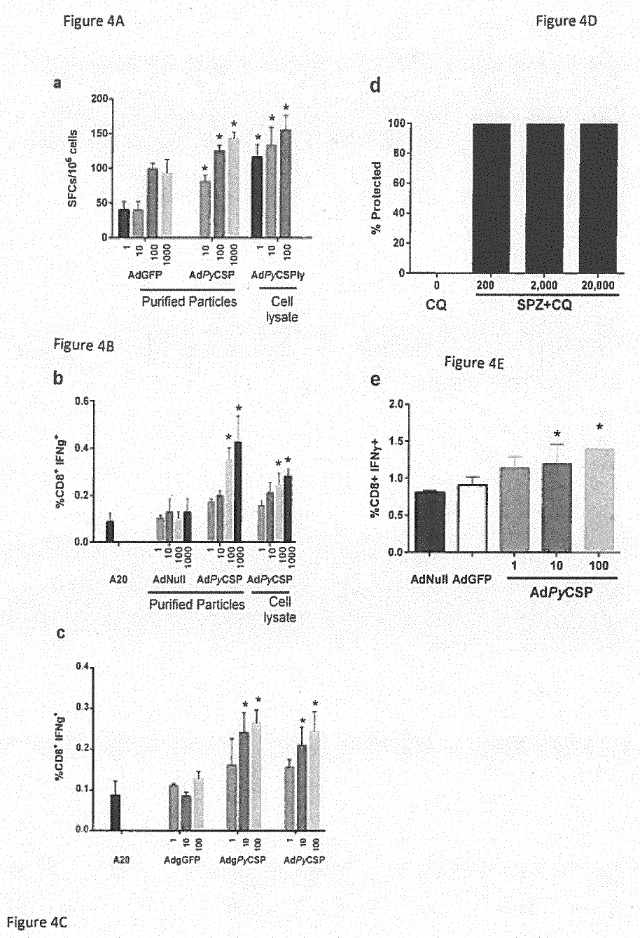

To determine whether lower-frequency T cell responses from mice immunized with sporozoite vaccines could be identified using our approach, we assayed CD8+ T cell responses specific for PyCSP from mice immunized with protective regimens of RAS and SPZ+CQ. PyCSP was selected as the test antigen because it is the most well-characterized target of T cell responses from mice immunized with these regimens. First, splenocytes were assayed from mice immunized with a highly protective three-dose regimen of RAS for the presence of PyCSP-specific T cells. PyCSP-specific T cells were able to be recalled in splenocytes from these mice using AdPyCSP-infected A20 cells in both ELISpot (FIG. 4A) and intracellular cytokine staining (ICS) assays (FIG. 4B). Low background responses were observed in the negative controls.

It was important to assess the degree of purity of the adenovector preparation necessary for the screen because if unpurified adenovectors were suitable, this would greatly simplify generation of the Ad-array. Accordingly, highly purified AdPyCSP (purified over three successive CsCl gradients) were compared with cell lysates containing unpurified recombinant adenovector. PyCSP-specific CD8+ T cell responses were detected with both purified and unpurified vectors using EliSpot (FIG. 4A) and ICS (FIG. 4B) assays. The results indicated that vector purification is not required to identify antigens that recall CD8+ T cell responses in mice immunized with RAS.

Ad-array vectors contain 25 bp-long att8 sequences flanking the transgene (FIG. 28), which are remnants of the recombinase cloning reaction. Ad-array vectors were directly compared with vaccine adenovectors, which do not carry the flanking attB sequences. The results indicate that the attB sequences did not inhibit the capacity to recall T cell responses in mice (FIG. 4C), indicating that Ad-array vectors are suitable for screening.

Mice immunized with a two-dose regimen of 200, 2,000, and 20,000 SPZ+CQ were completely protected from P. yoelii sporozoite challenge (FIG. 4D). FIG. 4E shows that PyCSP-specific T cells were induced by immunizing mice with a highly protective 2,000 SPZ+CQ regimen. Splenocytes from immunized mice had a high background of activated CD8+ T cells. When incubated with A20 cells infected with the negative control vectors AdNull and AdGFP, 0.8%-0.9% of the CD8+ T cells were activated. A20 cells infected with AdPyCSP recalled PyCSP-specific T cell responses that were more frequent than the negative controls. Statistically significant results were observed with MOIs of 10 and 100 ffu/cell. These data suggested that it would be possible to utilize our Ad-array technology to identify new antigen targets of protective T cell responses following immunization of mice with SPZ+CQ.

### Example 3: Identification of the Antigen Targets of CD8+ T Cells Induced Following Vaccination with Protective Regimens of SPZ+CQ

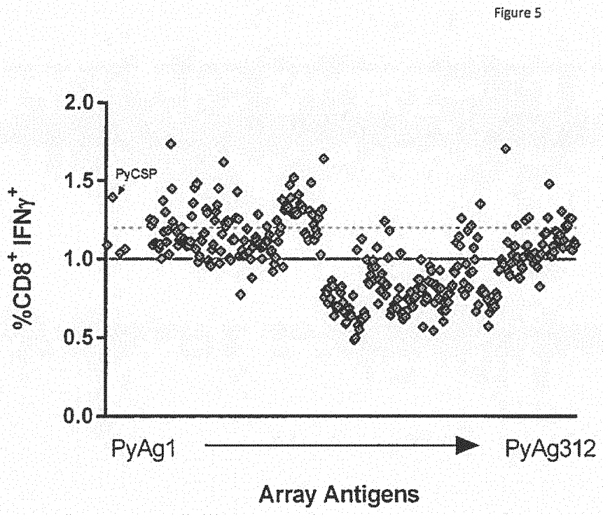

The 2,000 SPZ+CQ regimen was used to generate protective T cells for the identification of antigens. Splenocytes were harvested 2 weeks after the last sporozoite immunization. The full array was screened simultaneously, in triplicate, against these freshly isolated splenocytes by ICS to identify pre-erythrocytic stage antigens able to recall IFNγ-expressing CD8+ T cells. A20 cells infected with 100 ffu/cell AdgPyCSP were included as a positive control. Negative controls included uninfected A20 cells and A20 cells infected with 100 ffu/cell of AdNull and AdGFP vectors. The mean of the negative controls was 1% IFNγ-expressing CD8+ T cells (FIG. 5). Antigens with responses greater than 2 SD of the mean of the negative controls (>1.2% CD8+ IFNγ+ cells) were defined as positive hits in the screen. By this definition, 69 of the antigens in the array were positive and were targeted by CD8+ T cells induced in mice immunized with SPZ+CQ (FIG. 5). Thirteen of these antigens recalled higher-frequency CD8+ T cell responses than PyCSP. CD4+ T cell responses and tumor necrosis factor (TNF)-α and interleukin (IL)-2 cytokines were analyzed by ICS. CD4+ T cell responses were not observed in this system. CD8+ TNF-α-expressing T cells were observed and tended to mirror the CD8+ IFNα responses. Very low levels of IL-2-expressing cells were observed.

### Example 4: Identification of Protective Antigens

Since the SP2+CQ regimen induces protective T cell responses directed against antigens expressed in the pre-erythrocytic stages of the parasite life cycle, it was hypothesized that a subset of antigens identified in the SP2+CQ screen would induce protective immune responses when delivered using a potent vaccine regimen designed to optimize CD8+ T cell responses. The protective capacity of antigens was tested using a DNA prime-Ad5 boost regimen in BALB/c mice. Mice were immunized with 100 μg of DNA vector expressing the specific antigen and then boosted 6 weeks later with 1×10¹⁰ particle units (PUs) of an Ad5 vector expressing the same antigen. Two weeks after the Ad5 boost, mice were challenged with P. yoelii sporozoites and protection was monitored by microscopic examination of Giemsa-stained blood smears. Twenty-one percent (21%) of the PY00525 immunized mice were completely protected from sporozoite challenge, indicating that PY00525 can provide protection in mice. Twenty-one percent (21%) of the PY02793 immunized mice were completely protected from sporozoite challenge, indicating that PY02793 can provide protection in mice. Twenty-one percent (21%) of the PY03289 immunized mice were completely protected from sporozoite challenge, indicating that PY03289 can provide protection in mice. Thirty-six percent (36%) of the PY03674 immunized mice were completely protected from sporozoite challenge, indicating that PY03674 can provide protection in mice. The positive controls, which were immunized with PyCSP expressing DNA and Ad5 vectors in the same regimen, protected 100% of the mice. The negative controls, immunized with DNA and Ad5 Null vectors that did not express any transgene did not protect any mice. These data indicate that the antigen discovery system is capable of identifying protective antigens.

### Example 5: The P. falciparum Ortholog of PY03674 is Immunogenic in BALB/c Mice

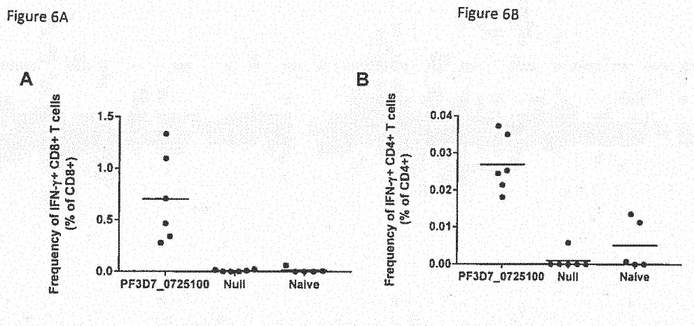

To begin evaluation of a selected pre-erythrocytic antigen as a vaccine candidate, the P. falciparum ortholog of PY03674 was cloned into a highly immunogenic and low seroprevalent gorilla adenovector (GC46) and tested immunogenicity in mice. PF3D7_0725100 (SEQ ID NO.: 17) was codon optimized for expression in mammals, synthesized, and used to produce GC46.PF3D7_0725100. BALB/c mice (n=6/group) were immunized with a single intramuscular (IM) administration of GC46. PF3D7_0725100 (1×10 PFU). GC46.Null immunized and naive mice were included as control groups. At 21 days post-immunization, mice were euthanized for T cell studies. Antigen-specific T cell responses were measured from splenocytes by flow cytometry after stimulation with overlapping peptide pools and staining for cytokines and cell surface markers. GC46. PF3D7_0725100 was immunogenic, inducing both antigen-specific CD8+ and CD4+ T cell responses (FIG. 6).

In particular, BALB/c mice were immunized with a single dose of 1×10⁹ PFU of GC46.PF3D7_0725100 by the intramuscular route with a 1 ml syringe and a 30G needle (Becton Dickinson Co., Franklin Lakes, N.J.). At 21 days post immunization, mice were euthanized for splenocyte harvest and assessment of immune responses by ICS and flow cytometry. Splenocytes from GC46.PF3D7_0725100 Immunized mice were harvest and plated at 2×10⁶ cells per well in a 96 well v-bottom plate. Cells were stimulated for 4 hours in the presence of 20 μg/mL brefeldin A (Sigma-Aldrich) with either 15-mer peptides for the PF3D7_0725100 antigen at 2 g/mL, overlapping by 10 amino acids (Mimotopes), or 1% DMSO as a negative control. Subsequently, cells were stained with Live/Dead™ Fixable Blue Dead Cell Stain Kit, for UV excitation (Invitrogen), surface stained with CD14 Phycoerythrin (PE) (clone Sa14-2, Life Technologies), CD19 Brilliant Violet 650 (clone 6D5, Biolegend), CD3 Alexa 700 (clone 17A2, Biolegend), CCR7 PerCPCy5.5 (done 4812, eBioscience), CD44 Pacific Blue (done IM7, Biolegend) and CD62L Brilliant Violet 786 (clone MEL-14, BD Biosciences), and permeabilized using Cytofix/Cytoperm reagent (BD Biosciences). Cells were then intracellularly stained with CD4 Brilliant Violet 605 (clone RM4-5, Biolegend), together with CD8 Horizon VS00 (clone 53-6.7), TNF Cy7PE (clone MP6-XT22), IFNα allophycocyanin (APC) (clone XMG1.2), and IL-2 FITC (done JES6-5H4) from BD Biosciences. To identify antigen-specific responses, data was acquired by flow cytometry and cells were gated on forward scatter (threshold), exclusion of aggregates, and subsequently to include singlets, viable cells, CD14-, CD3+, CD19-, CD3+, lymphocytes, and either CD4+ or CD8+ populations.

### Example 6: Identification of Protective and Immunogenic Antigens Using a Matrix Format

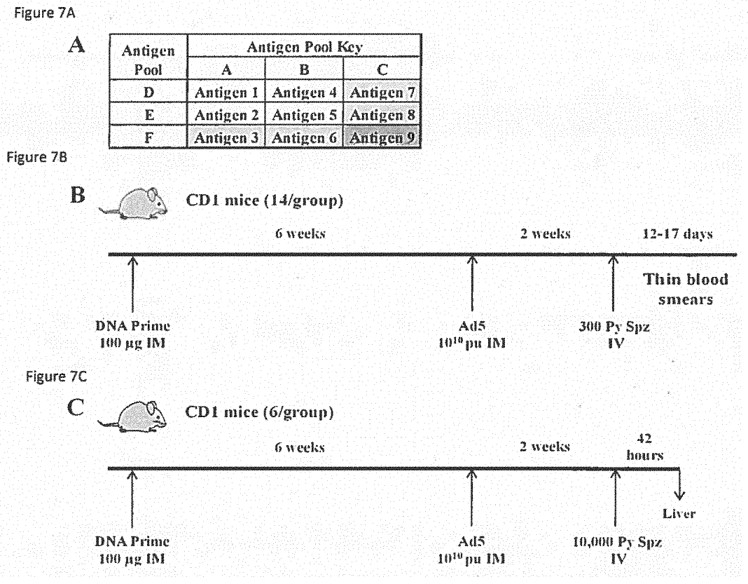

A consistent strategy was developed to screen protective antigens in mice against P. yoelii sporozoite challenge CD-1 outbred mice are immunized with DNA-prime/Ad5-boost vaccines expressing a combination of antigens in a matrix format, challenged by intravenous injection of P. yoelii sporozoites, and assessed for sterile protection by blood smear (FIG. 7).

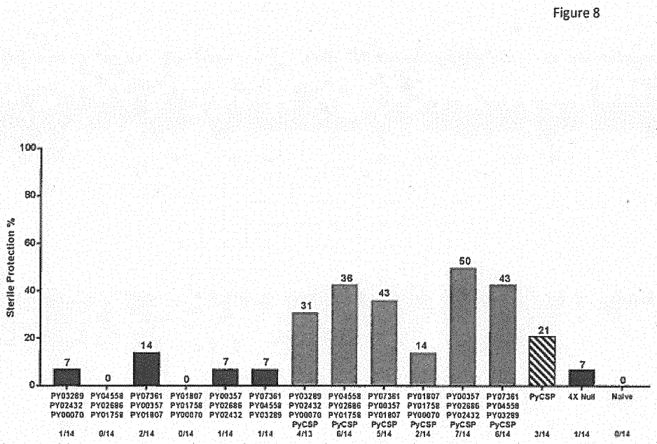

In an experiment using this strategy, low level protection was observed in all groups lacking PyCSP: no pool of three antigens without PyCSP exceeded the protection induced by PyCSP alone (FIG. 8). When combining antigen pools with PyCSP, five of six groups exhibited increased protection compared to PyCSP alone, and the maximal protection observed was 50% of mice. Setting aside potential interference among antigens for the present, these data suggest that none of the nine antigens evaluated may be as effective as the current gold standard, PyCSP, but that several of these antigens may be able to enhance protection in combination with PyCSP. By summing the number of protected mice for each antigen, we determined the following antigen hierarchy: PY00357>PY02686=PY07361>PY02432=PY04558>PY03289. Antigens PY00070, PY01758, and PY01807 were least protective. This experiment demonstrates that these antigens can enhance protection elicited by CSP when administered in combination.

Subsequently, additional experiments were performed using an identical format with different antigens to evaluate protective efficacy of additional antigens, and also to deconvolute protection elicited by combinations of antigens. Importantly, while the combination of PY03396 and PY05693 together in the absence of other antigens was not protective (0/14 mice protected), both antigens PY03396 and PY05693 were separately able to enhance protection of PY06306 (disclosed in US 20170232091A1) from 71% to 100% in both cases, demonstrating the ability of these antigens to work in combination with other vaccine antigens and enhance protective efficacy. This is important because multiple antigens may be combined to generate a successful subunit vaccine against P. falciparum and/or P. vivax malaria in humans.

1. A pharmaceutical composition comprising an immunologically effective amount of at least one antigenic polypeptide having an amino acid sequence that is at least 90% identical to an amino acid sequence selected from the group consisting of SEQ ID NO:14, SEQ ID NO:15, SEQ ID NO:16, SEQ ID NO:17, SEQ ID NO:40, NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, NO:61, SEQ ID NO:62, SEQ ID NO:63, SEQ ID NO:64, and SEQ ID NO:65, SEQ ID NO:66, SEQ ID NO:67, SEQ ID NO:68, SEQ ID NO:69, SEQ ID NO:70, NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, NO:91, SEQ ID NO:92, SEQ ID NO:93, SEQ ID NO:94, SEQ ID NO:95, SEQ ID NO:96 and SEQ ID NO:97, SEQ ID NO:98, SEQ ID NO:99, NO:100, SEQ ID NO:101, SEQ ID NO:102, SEQ ID NO:103, SEQ ID NO:104, SEQ ID NO:105 and SEQ ID NO:106, and a pharmaceutically acceptable carrier.

2. A pharmaceutical composition comprising a DNA expression vector encoding at least one antigenic polypeptide having an amino acid sequence that is at least 90% identical to an amino acid sequence selected from the group consisting of SEQ ID NO:14, SEQ ID NO:15, SEQ ID NO:16, SEQ ID NO:17, SEQ ID NO:40, NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, NO:61, SEQ ID NO:62, SEQ ID NO:63, SEQ ID NO:64, and SEQ ID NO:65, SEQ ID NO:66, SEQ ID NO:67, SEQ ID NO:68, SEQ ID NO:69, SEQ ID NO:70, NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, NO:91, SEQ ID NO:92, SEQ ID NO:93, SEQ ID NO:94, SEQ ID NO:95, SEQ ID NO:96 and SEQ ID NO:97, SEQ ID NO:98, SEQ ID NO:99, NO:100, SEQ ID NO:101, SEQ ID NO:102, SEQ ID NO:103, SEQ ID NO:104, SEQ ID NO:105 and SEQ ID NO:106, and a pharmaceutically acceptable carrier.

3. The composition of claim 2, wherein the DNA expression vector is a DNA plasmid, alphavirus, replicon, adenovirus, poxvirus, adenoassociated virus, cytomegalovirus, canine distemper virus, yellow fever virus, retrovirus, RNA replicons, DNA replicons, alphavirus replicon particles, Venezuelan Equine Encephalitis virus, Semliki Forest Virus or Sindbus Virus.

4. A method of inducing an immune response against Plasmodium falciparum comprising administering to a subject in need thereof an immunologically effective amount of a composition comprising at least one antigenic peptide that is at least 90% identical to an amino acid sequence selected from the group consisting of SEQ ID NO:14, SEQ ID NO:15, SEQ ID NO:16, SEQ ID NO:17, SEQ ID NO:40, NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, NO:51, SEQ ID NO:52, SEQ ID NO:53, SEQ ID NO:54, SEQ ID NO:55, SEQ-ID NO:56, SEQ ID NO:57, SEQ ID NO:58, SEQ ID NO:59, SEQ ID NO:60, NO:61, SEQ ID NO:62, SEQ ID NO:63, SEQ ID NO:64, and SEQ ID NO:65, SEQ ID NO:66, SEQ ID NO:67, SEQ ID NO:68, SEQ ID NO:69, SEQ ID NO:70, NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, NO:91, SEQ ID NO:92, SEQ ID NO:93, SEQ ID NO:94, SEQ ID NO:95, SEQ ID NO:96 and SEQ ID NO:97, SEQ ID NO:98, SEQ ID NO:99, NO:100, SEQ ID NO:101, SEQ ID NO:102, SEQ ID NO:103, SEQ ID NO:104, SEQ ID NO:105 and SEQ ID NO:106, and a pharmaceutically acceptable carrier.

5. The method of claim 4, wherein administering the peptide to the subject comprises administering a DNA expression vector encoding the peptide.

6. The method of claim 5, wherein the DNA expression vector is a DNA plasmid, alphavirus, replicon, adenovirus, poxvirus, adenoassociated virus, cytomegalovirus, canine distemper virus, yellow fever virus, retrovirus, RNA replicons, DNA replicons, alphavirus replicon particles, Venezuelan Equine Encephalitis virus, Semliki Forest Virus or Sindbus Virus.

7. The method of claim 4, wherein the immune response comprises inducing an antibody response.

8. The method of claim 4, wherein the immune response is a cellular immune response that comprises inducing a CD8⁺ T cell response.

9. The method of claim 8, wherein the induced CD8⁺ T cell response comprises CD8⁺ T cells expressing higher levels of interferon gamma (IFNγ) compared to CD8⁺ T cells that have not induced.

10. The method of claim 4, wherein the method further comprises administering a booster composition to the subject, wherein the booster composition comprises at least one antigenic polypeptide having an amino acid sequence that is at least 90% identical to an amino acid sequence selected from the group consisting of any of SEQ ID NO:14, SEQ ID NO:15, SEQ ID NO:16, SEQ ID NO:17, SEQ ID NO:40, NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, NO:61, SEQ ID NO:62, SEQ ID NO:63, SEQ ID NO:64, and SEQ ID NO:65, SEQ ID NO:66, SEQ ID NO:67, SEQ ID NO:68, SEQ ID NO:69, SEQ ID NO:70, NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, NO:91, SEQ ID NO:92, SEQ ID NO:93, SEQ ID NO:94, SEQ ID NO:95, SEQ ID NO:96 and SEQ ID NO:97, SEQ ID NO:98, SEQ ID NO:99, NO:100, SEQ ID NO:101, SEQ ID NO:102, SEQ ID NO:103, SEQ ID NO:104, SEQ ID NO:105 and SEQ ID NO:106, and a pharmaceutically acceptable carrier.

11. The method of claim 8, wherein the DNA expression vector in the booster composition is a DNA plasmid, alphavirus, replicon, adenovirus, poxvirus, adenoassociated virus, cytomegalovirus, canine distemper virus, yellow fever virus, retrovirus, RNA replicons, DNA replicons, alphavirus replicon particles, Venezuelan Equine Encephalitis virus, Semliki Forest Virus or Sindbus Virus.

12. The method of claim 5, wherein the immune response comprises inducing an antibody response.

13. The method of claim 6, wherein the immune response comprises inducing an antibody response.

14. The method of claim 5 wherein the immune response is a cellular immune response that comprises inducing a CD8⁺ T cell response.

15. The method of claim 6 wherein the immune response is a cellular immune response that comprises inducing a CD8⁺ T cell response.

16. The method of claim 14, wherein the induced CD8⁺ T cell response comprises CD8⁺ T cells expressing higher levels of interferon gamma (IFNγ) compared to CD8⁺ T cells that have not induced.

17. The method of claim 15, wherein the induced CD8⁺ T cell response comprises CD8⁺ T cells expressing higher levels of interferon gamma (IFNγ) compared to CD8⁺ T cells that have not induced.

18. The method of claim 10 wherein the administration of the booster composition to the subject comprises administering a DNA expression vector encoding the at least one antigenic polypeptide.
